SEQUENCE LISTING (39 sequences)

SEQ ID NO: 1 - protein, 106 aa, synthetic construct
  REGION 1..106: note = Variable light chain
DIQMTQSPSSLSASVGDRVTITCKGSQDISNYLAWYQQKPGKAPKLLIYNTDILHTGVPSRFSGSGSGTDFTFTISSLQPEDIATYYCYQYDTGYTFGQGTKVEIK

SEQ ID NO: 2 - protein, 121 aa, synthetic construct
  REGION 1..121: note = Variable heavy chain
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 3 - protein, 213 aa, synthetic construct
  REGION 1..213: note = Full light chain
DIQMTQSPSSLSASVGDRVTITCKGSQDISNYLAWYQQKPGKAPKLLIYNTDILHTGVPSRFSGSGSGTDFTFTISSLQPEDIATYYCYQYDTGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 451 aa, synthetic construct
  REGION 1..451: note = Full heavy chain
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 589 aa, synthetic construct
  REGION 1..589: note = Heavy chain +linker+IL2 (E61K)
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSTKKTQLQLEHLLLDLQMILNGINNYKNPKLTRMLTFKFYMPKKATELKHLQCLEKELKPLEEVLNLAQSKNFHLRPRDLISNINVIVLELKGSETTFMCEYADETATIVEFLNRWITFCQSIISTLT

SEQ ID NO: 6 - protein, 5 aa, synthetic construct
  REGION 1..5: note = HCDR1
DFGMN

SEQ ID NO: 7 - protein, 17 aa, synthetic construct
  REGION 1..17: note = HCDR2
LIYYDGSNKFYADSVKG

SEQ ID NO: 8 - protein, 12 aa, synthetic construct
  REGION 1..12: note = HCDR3
PHYDGYYHFFDS

SEQ ID NO: 9 - protein, 11 aa, synthetic construct
  REGION 1..11: note = LCDR1
KGSQDISNYLA

SEQ ID NO: 10 - protein, 7 aa, synthetic construct
  REGION 1..7: note = LCDR2
NTDILHT

SEQ ID NO: 11 - protein, 8 aa, synthetic construct
  REGION 1..8: note = LCDR3
YQYDTGYT

SEQ ID NO: 12 - protein, 129 aa, synthetic construct
  REGION 1..129: note = Mutant IL2 (E61K)
SSTKKTQLQLEHLLLDLQMILNGINNYKNPKLTRMLTFKFYMPKKATELKHLQCLEKELKPLEEVLNLAQSKNFHLRPRDLISNINVIVLELKGSETTFMCEYADETATIVEFLNRWITFCQSIISTLT

SEQ ID NO: 13 - protein, 5 aa, synthetic construct
  REGION 1..5: note = Single linker
GGGGS

SEQ ID NO: 14 - protein, 9 aa, synthetic construct
  REGION 1..9: note = Linker (2XG4S)
GGGGSGGGG

SEQ ID NO: 15 - protein, 4 aa, synthetic construct
  REGION 1..4: note = IL2 residues removed
APTS

SEQ ID NO: 16 - protein, 133 aa, synthetic construct [sequence omitted]

SEQ ID NO: 17 - protein, 129 aa, synthetic construct
  REGION 1..129: note = IL20212 (E61Q)
SSTKKTQLQLEHLLLDLQMILNGINNYKNPKLTRMLTFKFYMPKKATELKHLQCLEQELKPLEEVLNLAQSKNFHLRPRDLISNINVIVLELKGSETTFMCEYADETATIVEFLNRWITFCQSIISTLT

SEQ ID NO: 18 - protein, 129 aa, synthetic construct
  REGION 1..129: note = IL20212 (E61A)
SSTKKTQLQLEHLLLDLQMILNGINNYKNPKLTRMLTFKFYMPKKATELKHLQCLEAELKPLEEVLNLAQSKNFHLRPRDLISNINVIVLELKGSETTFMCEYADETATIVEFLNRWITFCQSIISTLT

SEQ ID NO: 19 - protein, 129 aa, synthetic construct
  REGION 1..129: note = IL20212 (E62A)
SSTKKTQLQLEHLLLDLQMILNGINNYKNPKLTRMLTFKFYMPKKATELKHLQCLEEALKPLEEVLNLAQSKNFHLRPRDLISNINVIVLELKGSETTFMCEYADETATIVEFLNRWITFCQSIISTLT

SEQ ID NO: 20 - protein, 129 aa, synthetic construct
  REGION 1..129: note = IL20212 (E62K)
SSTKKTQLQLEHLLLDLQMILNGINNYKNPKLTRMLTFKFYMPKKATELKHLQCLEEKLKPLEEVLNLAQSKNFHLRPRDLISNINVIVLELKGSETTFMCEYADETATIVEFLNRWITFCQSIISTLT

SEQ ID NO: 21 - protein, 129 aa, synthetic construct
  REGION 1..129: note = IL20212 (E62Q)
SSTKKTQLQLEHLLLDLQMILNGINNYKNPKLTRMLTFKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 22 - protein, 129 aa, synthetic construct
  REGION 1..129: note = IL20212 (H16A)
SSTKKTQLQLEALLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 129 aa, synthetic construct
  REGION 1..129: note = IL20212 (H16E)
SSTKKTQLQLEELLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 129 aa, synthetic construct
  REGION 1..129: note = IL20212 (H16Q)
SSTKKTQLQLEQLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 25 - protein, 129 aa, synthetic construct
  REGION 1..129: note = IL20212 (D20A)
SSTKKTQLQLEHLLLALQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 26 - protein, 129 aa, synthetic construct
  REGION 1..129: note = IL20212 (D20T)
SSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 129 aa, synthetic construct
  REGION 1..129: note = IL20212 (F42A)
SSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 129 aa, synthetic construct
  REGION 1..129: note = IL20212 (F42K)
SSTKKTQLQLEHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 29 - protein, 129 aa, synthetic construct
  REGION 1..129: note = IL20212 (R38A)
SSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - protein, 129 aa, synthetic construct
  REGION 1..129: note = IL20212 (R38E)
SSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - protein, 129 aa, synthetic construct
  REGION 1..129: note = IL20212 (L72G)
SSTKKTQLQLEHLLLDLQMILNGINNYKNPKLTRMLTFKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 32 - protein, 129 aa, synthetic construct
  REGION 1..129: note = IL20212 (L72A)
SSTKKTQLQLEHLLLDLQMILNGINNYKNPKLTRMLTFKFYMPKKATELKHLQCLEEELKPLEEVLNAAQSKNFHLRPRDLISNINVIVLELKGSETTFMCEYADETATIVEFLNRWITFCQSIISTLT

SEQ ID NO: 33 - protein, 129 aa, synthetic construct
  REGION 1..129: note = IL20212 (N88R)
SSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 34 - protein, 129 aa, synthetic construct
  REGION 1..129: note = IL20212 (N88A)
SSTKKTQLQLEHLLLDLQMILNGINNYKNPKLTRMLTFKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 35 - protein, 129 aa, synthetic construct
  REGION 1..129: note = IL20212 (C125A)
SSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 36 - protein, 129 aa, synthetic construct
  REGION 1..129: note = IL20212 (C125S)
SSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 37 - protein, 129 aa, synthetic construct
  REGION 1..129: note = IL20212 (C125V)
SSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFVQSIISTLT

SEQ ID NO: 38 - protein, 129 aa, synthetic construct
  REGION 1..129: note = IL20212 (Q126A)
SSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCASIISTLT

SEQ ID NO: 39 - protein, 129 aa, synthetic construct
  REGION 1..129: note = IL20212 (Q126T)
SSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCTSIISTLT

(19) United States
(12) Patent Application Publication
(10) Pub. No.: US 2025/0249093 A1
(43) Pub. Date: Aug. 7, 2025

(54) CYTOKINE ENGAGERS AND USES THEREOF

(71) Applicant: AstraZeneca AB, Södertälje (SE)

(72) Inventors: Matthew ELDER, Cambridge (GB); Aidan RILEY, Cambridge (GB); James STEINHARDT, Gaithersburg, MD (US); Simon DOVEDI, Cambridge (GB); Saso CEMERSKI, Giathersburg, MD (US); Gianluca CARLESSO, Gaithersburg, MD (US); Jeffrey RIGGS, Gaithersburg, MD (US); Carolina OROZCO, Cambridge (GB); Andie COLLINSON, Cambridge (GB); Jerome MASTIO, Cambridge (GB); Sin Lih TAN, Cambridge (GB); Bruno FREDERICO, Cambridge (GB); Hung-Chang CHEN, Cambridge (GB)

(21) Appl. No.: 19/042,085

(22) Filed: Jan. 31, 2025

Related U.S. Application Data
(60) Provisional application No. 63/627,948, filed on Feb. 1, 2024.

(51) Int. Cl. A61K 39/395; A61K 38/20; A61K 39/00; A61P 37/04; C07K 14/55; C07K 16/28

(52) U.S. Cl. CPC A61K 39/3955; A61K 38/2013; A61P 37/04; C07K 14/55; C07K 16/2815; A61K 2039/505; C07K 2317/92

(57) ABSTRACT

The disclosure provides a cytokine engager comprising an antibody that specifically bind human CD8, and wherein the antibody is fused to a cytokine (e.g., IL-2). Compositions and methods of administering the compositions to preferentially expand T cells and treat cancer are also provided.

### REFERENCE TO SEQUENCE LISTING SUBMITTED ELECTRONICALLY VIA EFS WEB

The content of the electronically submitted sequence listing (CD8IL2-100-US-PSP_SequenceListing_ST26.xml; Size: 53,713 bytes; and Date of Creation: Feb. 1, 2024) submitted in this application is incorporated herein by reference in its entirety.

### BACKGROUND OF THE DISCLOSURE

CD8+ T cell activation and differentiation is in large part controlled by soluble immunomodulatory proteins such as cytokines. Biological activity of cytokines is mediated by binding to their respective cytokine receptors on the cell surface, typically with very high affinity, resulting in their ability to potently stimulate signal transduction downstream of their receptors triggering various cellular processes that regulate immune cell phenotype and function. Cytokines typically have pleiotropic effects, causing multiple downstream cellular events such as activation, proliferation, survival, apoptosis, and secretion of other immunomodulatory proteins. In addition, because their receptors are expressed on multiple immune cell subsets, cytokines act not only on CD8+ T cells but also on other immune and non-immune cells that express their receptors.

For example, interleukin-2 (IL-2) is a cytokine that regulates many lymphocyte subsets, including alpha beta CD4+, CD8+ and regulatory T (Treg) cells, and various innate and innate-like lymphocytes such as NK cells, NK T cells, gamma delta T cells (Tγδ) cells, and innate lymphoid cells (ILC1, ILC2, and ILC3 cells). IL-2 can signal by binding with an intermediate affinity to a receptor complex consisting of IL-2Rβ (CD122) and IL-2Rγ (CD132) subunits (IL-2Rβγ, intermediate affinity receptor), both of which are required and sufficient to trigger downstream signaling in immune cells. In addition, IL-2 binds with high affinity to a receptor complex consisting of IL-2Rα (CD25), IL-2Rβ (CD122), and IL-2Rγ (CD132) subunits (IL-2αβγ, high affinity receptor) (Stauber et al, Proc Natl Acad Sci USA. 2006 Feb. 21; 103(8):2788-93). IL-2Rα expression is restricted to CD4+ Treg cells, activated T lymphocytes, and ILC2 and ILC3 cells, making these subsets the most sensitive to IL-2 signaling. IL-2Rβ and IL-2Rγ subunits are shared with another related cytokine, IL-15, and IL-2Rγ subunit is shared among other common gamma chain cytokines (IL-4, IL-7, IL-9, and IL-21). Most innate and innate-like lymphocytes including NK cells, NK T cells, Tγδ cells, and ILC1, ILC2, and ILC3 cells express high levels of IL-2Rβ (ImmGen consortium; Heng T S et al, Immunological Genome Project Consortium. Nat Immunol. 2008 October; 9(10):1091-4), which also makes them sensitive to both IL-2 and IL-15 cytokines.

IL-2 is a potent cytokine that stimulates T and NK cell proliferation through either a heterotrimeric IL-2 receptor (IL-2R) composed of CD25, CD122 and CD132, or a heterodimeric IL-2 receptor composed of only CD122 and CD132. Both forms of the IL-2R are potent mediators of T cell survival, proliferation, and overall activation status. IL-2 is generally produced by T cells and NK cells upon activation and mediates signaling in cis and trans in the local microenvironment. IL-2R signaling can induce differentiation of naïve T cells into effector and memory T cells and can also stimulate suppressive Tregs. Although the trimeric form of the IL-2R has a higher affinity for IL-2 than the dimeric form, both are reasonably high affinity and cause rapid receptor mediated internalization and degradation, resulting in an extremely short half-life. Recombinant human IL-2 (rhIL-2, unguided IL-2) is used clinically to treat renal cell carcinoma and malignant melanoma; however, it is associated with severe toxicity. Vascular leak syndrome is a major toxicity concern for cancer patients treated with unguided IL-2 due in-part to the effects of IL-2 signaling on endothelial cells that express the high affinity IL-2R.

Thus, there is a need for CD8-binding cytokine engagers that can specifically target activating molecules to CD8⁺ T cells to increase the potency and selectivity of cytotoxic T cell responses. The present disclosure addresses this need and others.

### SUMMARY OF THE DISCLOSURE

The present disclosure is directed to cytokine engagers comprising an antigen binding molecule that binds CD8 fused to an immunomodulatory peptide. The present disclosure provides methods of modulating immune cell function by contacting the immune cell with the cytokine engagers of the present disclosure. In addition, the disclosure also provides polynucleotides encoding the disclosed cytokine engagers, and vectors and host cells comprising such polynucleotides. The present disclosure further provides methods for producing the cytokine engagers, pharmaceutical compositions comprising the same, and uses thereof.

Some aspects of the present disclosure are directed to a cytokine engager comprising an antibody that specifically binds cluster of differentiation 8 (CD8; also referred to herein as a CD8 co-receptor), wherein the antibody is linked to a cytokine (e.g., IL-2 or a mutant thereof).

In some aspects, the antibody specifically binds to the CD8a chain of CD8.

In some aspects, the antibody comprises: a heavy chain complementarity determining region (HCDR) 1, a HCDR2, and a HCDR3, comprising the amino acid sequence of SEQ ID NO: 6, SEQ ID NO: 7, and SEQ ID NO: 8, respectively; and a light chain complementarity determining region (LCDR) 1, a LCDR2, and a LCDR3, comprising the amino acid sequence of SEQ ID NO: 9, SEQ ID NO: 10, and SEQ ID NO: 11, respectively.

In some aspects, the antibody comprises a first heavy chain, a second heavy chain, and two light chains, and wherein: either the first heavy chain or the second heavy chain of the antibody is fused at the C-terminus to the cytokine; or the first heavy chain is fused at the C-terminus to the cytokine and the second heavy chain of the antibody is fused at the C-terminus to a second cytokine.

In some aspects, the antibody is fused to the cytokine and/or the second cytokine via a linker comprising SEQ ID NO: 13.

In some aspects, the antibody is fused to the cytokine and/or the second cytokine via a linker comprising SEQ ID NO: 14.

In some aspects, the cytokine and/or the second cytokine is interleukin-2 (IL-2).

In some aspects, the cytokine and/or the second cytokine is each an IL-2 mutant comprising one or more of the following substitutions: E61K, E61Q, E61A, E62A, E62K, E62Q, H16A, H16E, H16Q, D20A, D20T, F42A, F42K, R38A, R38E, L72G, L72A, N88R, N88A, C125A, C125S, C125V, Q126A, or Q126T.

In some aspects, the cytokine and/or the second cytokine is an IL-2 comprising an E61K substitution.

In some aspects, the first four amino acids of the IL-2 are deleted as compared to the wild-type sequence.

In some aspects, the IL-2 does not comprise SEQ ID NO: 15.

In some aspects, the IL-2 comprises a sequence according to any one of SEQ ID NOs: 12 or 17-39.

In some aspects, the antibody comprises a variable heavy chain and a variable light chain that is at least 90%, at least 91% at least 92%, at least 93%, at least 94%, at least 95%, at least 96%, at least 97%, at least 98%, or at least 99% identical to SEQ ID NO: 2 and SEQ ID NO: 1, respectively. In some aspects, the antibody comprises a variable heavy chain and a variable light chain comprising the amino acid sequence of SEQ ID NO: 2 and SEQ ID NO: 1, respectively.

In some aspects, the antibody comprises an Fc region from an IgG antibody. In some aspects, the Fc region is from an IgG1 antibody.

In some aspects, the antibody comprises an Fc region comprising the mutations L234F, L235E, and P331S, wherein the numbering is according to the EU index as in Kabat.

In some aspects, each heavy chain of the antibody comprises SEQ ID NO: 4 and each light chain of the antibody comprises SEQ ID NO: 3. In some aspects, each heavy chain of the antibody comprises SEQ ID NO: 5 and each light chain of the antibody comprises SEQ ID NO: 3.

In some aspects, the cytokine engager binds to CD8⁺ T cells and promotes proliferation of the CD8⁺ T cells. In some aspects, the CD8⁺ T cells are cytotoxic CD8⁺ T cells.

In some aspects, the cytokine engager promotes the expression of CD69 of CD8⁺ T cells. In some aspects, the cytokine engager induces the expression of CD69 in CD8⁺ T cells 2-fold, 5-fold, or greater than 5-fold higher than the expression of CD69 in NK cells.

Some aspects of the present disclosure are directed to a pharmaceutical composition comprising the cytokine engager as described herein and a pharmaceutically acceptable carrier.

Some aspects of the present disclosure are directed to an isolated nucleic acid sequence encoding a cytokine engager as described herein. Some aspects of the present disclosure as directed to a vector comprising the isolated nucleic acid sequence.

Some aspects of the present disclosure are directed to a method of expanding T cells comprising contacting a cytokine engager or a pharmaceutical composition, as described herein, to a population of T cells. In some aspects, the cytokine engager is administered to a subject in need. In some aspects, the population of T cells is endogenous to the subject. In some aspects, the subject has cancer. In some aspects, the CD8⁺ T cells preferentially expand compared to CD4⁺ T cells. In some aspects, the CD8⁺ T cells preferentially expand compared to NK cells. In some aspects, the expansion is compared to contacting SEQ ID NO: 12 to a population of T cells.

Some aspects of the present disclosure are directed to a cytokine engager or pharmaceutical composition as describe herein for use as a medicament.

Some aspects of the present disclosure are directed to a cytokine engager or pharmaceutical composition as describe herein for use in the treatment of cancer.

Some aspects of the present disclosure are directed to a method of treating cancer comprising administering to a subject in need thereof an effective amount of a cytokine engager or pharmaceutical composition as describe herein. In some aspects, the cancer is melanoma, renal cell carcinoma, gastric, or non-small cell lung cancer.

### BRIEF DESCRIPTION OF THE DRAWINGS

FIGS. 1A-1D show the response of Tregs, CD8+ T cells, NK cells, and CD4+ T cells to unguided IL-2, NIP228-IL2 (E61K) (IL20093), and anti-CD8a antibody-IL-2 (E61K) (IL20212). The proliferative response of each cell type to increasing concentrations of unguided IL-2 (FIG. 1A), IL20093 (FIG. 1B), and IL20212 (FIG. 1C) was assessed (shown as % proliferation relative to maximum unguided IL-2 response), and IFNγ release was also determined by ELISA (FIG. 1D).

FIGS. 2A-2B show the effects of increasing concentrations of unguided IL-2, IL20212, IL20093, or a no IL2 control on 2D cytolysis (FIG. 2A) and 3D cytolysis (FIG. 2B) of an OE21 oesophageal cancer cell line engineered to express pp65 antigen.

FIGS. 3A-3C show the effects of NIP228 IgG control or anti-CD8-IL2 (IL20212) treatment (at either 0.1, 1, or 3 mg/kg on day 6 and 13) on pp65-expressing OE21 tumor growth in NSG immunodeficient mice engrafted with 1e6 pp65+CD8+ T cells injected on day 2 (multi-donor). Fc-block treatment was administered on day 5 and 12. Tumor volume across the duration of the study is shown in FIG. 3A. The total cell number of CD8+ T cells in the blood (shown as cells per μL blood) and tumor (shown as cells per mm3) for each treatment is reported in FIG. 3B. FIG. 3C shows the frequency of TEMRA (effector memory RA cells) as % of CD8+ T cells and TOX (thymocyte selection-associated HMG BOX) expression in CD8+ T cells (shown as mean fluorescent intensity) in the tumor for each treatment group.

FIGS. 4A-4E show the expression of CD8α and CD8β in CD8+ T cells and NK cells at steady-state (shown as the proportion % positive) (FIG. 4A), gMFI (FIG. 4B), and surface antigen density (absolute number) (FIG. 4C). The expression of CD8α (FIG. 4D) and CD69 (FIG. 4E) in CD8+ T cells and NK cells in response to either unguided IL-2, NIP228-IL2, or CD8-IL2 treatment over time shown as gMFI.

FIGS. 5A-5C show the effects of increasing concentrations of unguided IL-2, anti-CD8-IL2, anti-COMP1, and COMP2 (IL-2Rb selective IL2 agonist) treatment on CD8⁺ T cell granzyme B (GrzB) expression (FIG. 5A; shown as % GrzB expression of CD8+ T cells relative to Treg-induced suppression of CD3-stimulated CD8+ T cells), CD8+ T cell IFNγ expression (FIG. 5B; shown as % IFNγ expression of CD8+ T cells relative to Treg-induced suppression of CD3-stimulated CD8+ T cells), and CD8+ T cell proliferation (FIG. 5C; shown as % proliferation of CD8+ T cells relative to Treg-induced suppression of CD3-stimulated CD8+ T cells).

FIGS. 6A-6E show the frequency of CD8+ T cells (FIG. 6A), pp65+CD8+ T cells (FIG. 6B), CD4+ T cells (FIG. 6C), Tregs (FIG. 6D), and NK cells (FIG. 6E) as % total live cells over time in response to anti-CD8-IL2, NIP228-IL2, unguided IL-2, or no drug treatments. % total live cells are shown before treatment, and after one (stim 1, duration 3 days), two (stim 2, duration 4 days), or three (stim 3, duration 3 days) doses of each drug.

FIGS. 7A-7B show the % on human CD8+ T cells (FIG. 7A) and cynomolgus CD8+ T cells (FIG. 7B) positive for pSTAT5 in response to increasing concentrations of anti-CD8-IL2 treatment.

FIGS. 8A-8B show expression of CD8α and CD8β across various cell types. CD8α (FIG. 8A) and CD8β (FIG. 8B) RNA transcript levels (normalized transcript per million) were compared from an HPA (human protein atlas) dataset across various cell types.

FIGS. 9A-9C show the response of Tregs, CD8+ T cells, NK cells, and CD4+ T cells to hOKT8-IL2 (E61K) (IL20226), and hOKT8 (altVL)-IL2 (E61K) (IL20251). The proliferative response of each cell type to increasing concentrations of IL20212 (FIG. 1C), IL20226 (FIG. 9A), and IL20251 (FIG. 9B) was assessed (shown as % proliferation relative to maximum unguided IL-2 response). IFNγ release (pg/mL) was also determined in comparison to unguided IL-2 and NIP228-IL2 (E61K) (IL20093) (FIG. 9D). E61K refers to a mutation in IL2.

FIGS. 10A-10B show the effects of anti-CD8-IL2, NIP228-IL2, unguided IL-2, and αCD3/αCD28 treatment on either CD8+ T cell proliferation (FIG. 10A) or CD4⁺ T cell proliferation (FIG. 10B) in the presence of suppressive tumor-associated macrophages (TAMs). Maximal T cell proliferation is displayed as αCD3/aCD28 treatment in the absence of TAMs.

FIGS. 11A-11B show the effects of anti-CD8-IL2 and unguided IL-2 treatment on IFNγ secretion in non-small cell lung cancer (NSCLC) tumor (FIG. 11A) and anti-CD8-IL2, unguided IL-2, and MEDI5752 treatment on IFNγ secretion in renal cell carcinoma (KIRC) tumor (FIG. 11B).

FIGS. 12A-12C show the effects of monovalent anti-CD8 diabody with either F42K-IL2 mutein or E61K-IL2 mutein on IFNγ secretion (FIG. 12A), IL-6 secretion (FIG. 12B), and TNFα secretion (FIG. 12C) from PBMC at three different concentrations.

FIGS. 13A-13C compare the effects of unguided IL-2, monovalent anti-CD8-E61K-IL2, and bivalent anti-CD8-E61K on IFNγ secretion (FIG. 13A), IL-6 secretion (FIG. 13B), and TNFα secretion (FIG. 13C) from PBMC at increasing concentrations.

FIGS. 14A-14D show a comparison of bivalent anti-CD8-IL2 (IL20212), bivalent anti-CD8-IL2 (IL20226), monovalent anti-CD8-IL2 (IL20245), monovalent anti-CD8-IL2 (IL20246) on % pSTAT5 (FIG. 14A), % CD69 (FIG. 14B), % CD25 (FIG. 14C) expression, and proliferation of CD8+ T cells (FIG. 14D). 2xG4S indicates two repeats of a Gly-Gly-Gly-Gly-Ser linker between the IL2 and anti-CD8 antibody, and 3xG4S indicates three repeats of the same.

FIGS. 15A-15E show the results of an in vivo NSG mouse study to determine tolerance of various anti-CD8-IL2 molecules. FIG. 15A shows a schematic outlining the NSG mouse study, wherein on day 0 mice were injected subcutaneously (s.c.) with pp65-expressing OE21 tumor cells, and on day 7 injected intravenously (i.v.) with 1e6 pp65+CD8+ T cells. An Fc block (20 mg/kg) was administered i.p. to a first mouse on days 11 and 18, and to a second and third mice on days 12 and 19. The test/drug substance (3 mg/kg) was administered i.p. to the first mouse on days 12 and 19, and to the second and third mice on days 13 and 20. The study endpoint was day 27. Bodyweight loss (calculated as a % of maximum bodyweight) was measured for mice treated with IL20226 (bivalent anti-CD8-IL2 with a 2xG4S linker) (FIG. 15B), IL20245 (monovalent anti-CD8-IL2 with a 2xG4S linker) (FIG. 15C), and IL20246 (monovalent anti-CD8-IL2 with a 3xG4S linker) (FIG. 15D). FIG. 15E shows the % bodyweight loss for mice treated with either bivalent anti-CD8-IL2 (IL20212) or bivalent anti-CD8-IL2 (IL20226).

FIGS. 16A-16F show the results of an in vivo NSG mouse study to determine the effects of various concentrations of an anti-CD8-IL2 conjugate (IL20212) on tumor growth. FIG. 16A shows a schematic outlining the NSG mouse study, wherein on day 0 mice were injected subcutaneously (s.c.) with pp65-expressing OE21 tumor cells, and on day 3 injected intravenously (i.v.) with 1e6 pp65+CD8+ T cells. An Fc block (20 mg/kg) was administered i.p. on days 6 and 13, and IL20212 was administered i.p. on days 7 and 14 at a dose of 3, 0.8, 0.6, and 0.2 mg/kg, or an isotype control was administered (3 mg/kg). The study endpoint was day 28. Tumor volume was measured beginning at day 3 of the study for each of the isotype control, and IL20212 treatments at 3, 0.8, 0.6, and 0.2 mg/kg dosages, as shown in FIGS. 16B-16F, respectively.

FIGS. 17A-17G show the results of an in vivo NSG MHCI−/−/II−/− mouse study to determine the effects of anti-CD8-IL2 drug (IL20212) treatment on CD8+ T cell expansion. FIG. 17A shows a schematic outlining the study, wherein on day 0 mice were injected intravenously (i.v.) with PBMCs, and on days 13 and 20 administered an Fc block (20 mg/kg). IL20212 was administered intraperitoneally (1 mg/kg) on days 14 and 21). Blood draws were performed each day from day 14 through day 27 of the study. Blood draws on days 14 and 21 were performed 1-hour post-dosing, and on day 20 before the Fc block. Ki67 expression was measured by gMFI after the first and second doses of IL20212 at the timepoints shown for both CD4+ T cells (FIG. 17B) and CD8+ T cells (FIG. 17C). Absolute number of CD4+ and CD8+ T cells was also measured in parallel, as shown in FIGS. 17D-17E, respectively. Peripheral on-cell receptor occupancy was measured for both CD4+ T cells (FIG. 17F) and CD8+ T cells (FIG. 17G).

FIGS. 18A-18D show the results of an in vivo NSG mouse study to compare the effects of IL20212 to comparator modalities for tumor growth inhibition. FIG. 18A shows a schematic outlining the NSG mouse study, wherein on day 0 mice were injected subcutaneously (s.c.) with pp65-expressing OE21 tumor cells, and on day 3 injected intravenously (i.v.) with pp65+CD8+ T cell enriched PBMCs pre-expanded for 10 days in vitro (2.91×106 total cells). An Fc block (20 mg/kg) was administered i.p. on days 6 and 13, IL20212, comparator 1 (COMP1), comparator 2 (COMP2), unguided IL-2, or an isotype control were administered i.p. on days 7 and 14 (unguided IL-2 was administered on days 6-9 and 13-16). The study endpoint was day 28. Tumor volume was measured beginning at day 3 of the study for each of COMP1, COMP2, and unguided IL-2, as shown in FIGS. 18B-18D, respectively. Isotype control and IL20212 effects (0.8 and 0.6 mg/kg) on tumor volume are shown above in FIGS. 16B and 16D-16E.

FIG. 19A-19B. FIG. 19A shows a diagram of the general structure of a bivalent anti-CD8 cytokine engager described herein. FIG. 19B shows a diagram of the general structure of a monovalent anti-CD8 cytokine engager described herein.

### DETAILED DESCRIPTION OF DISCLOSURE

The present disclosure relates to cytokine engagers that specifically bind an epitope on cluster of differentiation 8 (CD8) (also referred to herein as a CD8-binding cytokine engager), wherein the cytokine engagers comprise an antibody that specifically binds CD8, wherein the antibody is fused to a cytokine. Some aspects of the present disclosure are directed to isolated nucleic acid sequences comprising a nucleotide sequence encoding a CD8-binding cytokine engager, wherein the cytokine engager comprises an antibody that specifically binds an epitope on human CD8. Some aspects of the present disclosure are directed to a host cell comprising the isolated nucleic acid sequence. Other aspects of the present disclosure are directed to cytokines (e.g., interleukin-2 (IL-2) and mutants thereof) fused to a CD8-binding cytokine engager. Further aspects of the present disclosure are directed to methods of treating a subject in need thereof comprising administering the isolated nucleic acid sequence, the cell, and/or the CD8-binding cytokine engager thereof to the subject. In some aspects, the subject is afflicted with a cancer, or a tumor derived from a cancer.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Units, prefixes, and symbols are denoted in their Systéme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally occurring IgG antibodies, the heavy chain constant region is comprised of a hinge and three domains, CH1, CH2 and CH3. In some antibodies, e.g., naturally occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the antibodies described herein are of the IgG1 subtype. Immunoglobulins, e.g., IgG1, exist in several allotypes, which differ from each other in at most a few amino acids. “Antibody” includes, by way of example, both naturally occurring and non-naturally occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen (e.g., human CD8). The antigen-binding function of an antibody can be performed by fragments of a full-length antibody. Examples of binding fragments encompassed within the term “antigen-binding portion” of an antibody, e.g., an anti-CD8 antibody and/or CD8-binding cytokine engager described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Antigen-binding portions can be produced by recombinant DNA techniques, or by enzymatic or chemical cleavage of intact immunoglobulins.

The term “Fc domain” or “Fe region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

As used herein, the term “cytokine” refers to a molecule that mediates and/or regulates a biological or cellular function or process (e.g., immunity, inflammation, and hematopoiesis). It can function as a soluble form or cell-surface associated to bind the “cytokine receptor” on target immune cells to activate signaling. “Cytokine receptor” as used here is the polypeptide on the cell surface that activates intracellular signaling upon binding the cytokine on the extracellular cell surface. The term “cytokine” as used herein includes “lymphokines,” “chemokines,” “monokines,” and “interleukins”. Examples of useful cytokines include, but are not limited to, GM-CSF, IL-1α, IL-10, IL-2, IL-3, IL-4, IL-5, IL-6, IL-7, IL-8, IL-10, IL-12, IL-15, IFN-α, IFN-β, IFN-γ, MIP-1α, MIP-1β, TGF-α, TNF-α, and TNF-β. A particular cytokine is IL-2.

“IL-2”, “IL2”, or “Interleukin-2” as used herein refers to any native, mature IL-2 that results from processing of an IL-2 precursor in a cell. The term includes IL-2 from any vertebrate source, including mammals such as primates (e.g., humans and cynomolgus or rhesus monkeys) and rodents (e.g., mice and rats), unless otherwise indicated. The term also includes naturally occurring variants of IL-2, such as splice variants or allelic variants. A nonlimiting exemplary human IL-2 amino acid sequence is shown, e.g., in GenBank Accession No. NP 000577.2. Proleukin® (aldesleukin) is a recombinant form of IL-2 marketed by Prometheus Laboratories, San Diego, Calif.).

“Modified IL-2” or “Mutant IL-2 Polypeptide” as used herein, refers to a polypeptide that differs from a wild type IL-2 amino acid sequence by a substitution of at least one amino acid position. refers to IL-2 polypeptide that has altered, increased, or reduced affinity to its receptor or a subunit thereof. For example, when such Modified IL-2 has decreased affinity, it will result in reduced biological activity of the mutant. Modified IL-2 with reduced or increased affinity to the receptor and/or one or more of its subunits and thereby activity can be obtained by introducing a small number of amino acid mutations or substitutions. The mutant IL-2 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-2Rβγ. Desired activity may also include improved biophysical properties compared to the wild type IL-2 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. Preferably, a mutation or mutations are introduced to the polypeptide to modify its activity.

As used herein, the term “affinity” refers to a measure of the strength of the binding of an antigen or target (such as an epitope) to its cognate binding domain (such as a paratope). As used herein, the term “avidity” refers to the overall stability of the complex between a population of epitopes and paratopes (i.e., antigens and antigen binding domains).

As used herein, the term “cytokine engager” refers to an antibody or antigen-binding fragment thereof which is fused to at least one cytokine molecule (e.g., an IL-2 molecule). The antibody may be any of those described herein (e.g., an anti-CD8 antibody), and the at least one cytokine may be fused directly, or by means of a linker (e.g., a peptide linker) or chemical conjugation to either the N- or C-terminus of the heavy or the light chain of the antibody. In some aspects, two or more cytokine molecules are fused to the antibody or antigen binding fragment thereof.

The term “epitope” refers to a site on an antigen (e.g., CD8) to which an immunoglobulin, or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “binds to the same epitope” with reference to two or more antigen-binding moieties means that the antigen-binding moieties bind to the same segment of amino acid residues. Antigen-binding moieties that “compete with another antibody for binding to a target” refer to antigen-binding moieties that inhibit (partially or completely) the binding of the other antibody to the target.

As used herein, the terms “specific binding,” “selective binding,” “selectively binds,” and “specifically binds,” refer to an antigen-binding moiety (e.g., an antibody) binding to an epitope on a predetermined antigen. Typically, the antigen-binding moiety (e.g., an antibody) (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., human CD8, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antigen-binding moiety (e.g., an antibody and/or cytokine engager) that “specifically binds to human CD8” refers to an antigen-binding moiety (e.g., an antibody and/or cytokine engager) that binds to human CD8 with a K_{D} of 10⁻⁷ M or less, such as approximately less than 10⁻⁸M, 10⁻⁹ M or 10⁻¹⁰ M or even lower.

The term “polypeptide,” as used herein, is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with, any of these terms. The term further includes polypeptides that have undergone post-translational or post-synthesis modifications, for example, conjugation of a palmitoyl group, glycosylation, acetylation, phosphorylation, amidation, derivatization by known protecting/blocking groups, disulfide bond formation, proteolytic cleavage, or modification by non-naturally occurring amino acids. The term “peptide,” as used herein, encompasses full-length peptides and fragments, variants or derivatives thereof. A “peptide” as used herein can be part of a fusion polypeptide comprising additional components such as, e.g., an albumin or PEG moiety, to increase half-life. A peptide as used herein can also be derivatized in a number of different ways. A peptide can comprise modifications including conjugation of a palmitoyl group. The term “nucleic acid molecule,” as used herein, is intended to include DNA molecules and RNA molecules. A nucleic acid molecule can be single-stranded or double-stranded and can be cDNA. A polypeptide of the present disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides with a defined three-dimensional structure are referred to as folded, and polypeptides that do not possess a defined three-dimensional structure, but rather can adopt many different conformations and are referred to as unfolded. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are termed homo- or heterodimers, homo- or heterotrimers etc.

The terms “polypeptide” and “protein” also refer to modified polypeptides/proteins wherein the post-expression modification is affected including without limitation glycosylation, acetylation, phosphorylation, amidation, derivatization by known protecting/blocking groups, proteolytic cleavage, or modification by non-naturally occurring amino acids.

The term “fusion protein” refers to a combination or conjugation of two or more proteins or polypeptides that results in a novel arrangement of proteins that do not normally exist naturally. The fusion protein is a result of covalent linkages of the two or more proteins or polypeptides. The two or more proteins that make up the fusion protein may be arranged in any configuration from aminoterminal end (“NH2”) to carboxy-terminal end (“COOH”).

“Substitution” or “mutation” refers to a change to the polypeptide backbone wherein an amino acid occurring in the wild type sequence of a polypeptide is substituted to another amino acid at the same position in the said polypeptide. In some aspects, a mutation or mutations are introduced to modify polypeptide's affinity to its receptor thereby altering its activity such that it becomes different from the affinity and activity of the wild type cognate polypeptide. Mutations can also improve polypeptide's biophysical properties. Genetic methods may include site-directed mutagenesis, PCR, gene synthesis and the like. It is contemplated that methods of altering the side chain group of an amino acid by methods other than genetic engineering, such as chemical modification, may also be useful.

“Conservative amino acid substitutions” refer to substitutions of an amino acid residue with an amino acid residue having a similar side chain. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in a CD8-binding cytokine engager (e.g., an anti-CD8 antibody comprising an IL-2 polypeptide) is replaced with another amino acid residue from the same side chain family.

The percent identity between two sequences is a function of the number of identical positions shared by the sequences (i.e., % homology=#of identical positions/total #of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available atwww.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The term “promoter,” as used herein, refers to a DNA sequence recognized by the machinery of a cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible expression by external signals or agents; such elements can be located in the 5′ or 3′ regions of the native gene. In some aspects, the promoter can be a constitutively active promoter, a cell-type specific promoter, or an inducible promoter.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses by inducing apoptosis or programmed cell death in the target cell. They were termed “natural killers” because NK cells do not require activation in order to kill a target cell. T-cells play a major role in cell-mediated-immunity. T-cell receptors (TCR) expressed on the surface of T cells differentiate T cells from other lymphocyte types. The thymus, a specialized organ of the immune system, is primarily responsible for T cell maturation. There are six types of T-cells, namely: Helper T-cells (e.g. CD4+ cells); Cytotoxic T-cells (also known as TC, cytotoxic T lymphocyte, CTL, T-killer cell, cytolytic T cell, CD8+ T-cells or killer T cell); Memory T-cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2R.p, CXCR3, and LFA-1, and show numerous functional attributes distinctive of memory cells); (ii) central memory TCM cells express L-selectin and the CCR7, they secrete IL-2, but not IFNy or IL-4, and (iii) effector memory TEM cells, however, do not express L-selectin or CCR7 but produce effector cytokines like IFNy and IL-4); Regulatory T-cells (Tregs, suppressor T cells, or CD4+CD25+ regulatory T cells); Natural Killer T-cells (NKT); and Gamma Delta T-cells.

“T cells” or “T lymphocytes” are immune cells that play a key role in the orchestration of immune responses in health and disease. Two major T cell subsets exist that have unique functions and properties: T cells that express the CD8 antigen (CD8⁺ T cells) are cytotoxic or killer T cells that can lyse target cells using the cytotoxic proteins such as granzymes and perforin; and T cells that express the CD4 antigen (CD4⁺ T cells) are helper T cells that are capable of regulating the function of many other immune cell types including that of CD8⁺ T cells, B cells, macrophages etc. Furthermore, CD4⁺ T cells are further subdivided into several subsets such as: T regulatory (Treg) cells that are capable of suppressing the immune response, and T helper 1 (Th1), T helper 2 (Th2), and T helper 17 (Th17) cells that regulate different types of immune responses by secreting immunomodulatory proteins such as cytokines. T cells recognize their targets via alpha beta T cell receptors that bind to unique antigen-specific motifs and this recognition mechanism is generally required in order to trigger their cytotoxic and cytokine-secreting functions. “Innate lymphocytes” can also exhibit properties of CD8⁺ and CD4⁺ T cells, such as the cytotoxic activity or the secretion of Th1, Th2, and Th17 cytokines. Some of these innate lymphocyte subsets include NK cells and ILC1, ILC2, and ILC3 cells; and innate-like T cells such as TyS cells; and NK T cells. Typically, these cells can rapidly respond to inflammatory stimuli from infected or injured tissues, such as immunomodulatory cytokines, but unlike alpha beta T cells, they can respond without the need to recognize antigen-specific patterns.

As used herein, the term “CD8 co-receptor” or “CD8” means the cell surface glycoprotein CD8, either as an alpha-alpha homodimer or an alpha-beta heterodimer. The CD8 alpha, or CD8α, is one of the two polypeptide chains that make up the CD8 glycoprotein. The CD8 beta, or CD8β is the second polypeptide chain of the CD8 glycoprotein. The combination of both alpha and beta chains contributes to the formation of the functional CD8 co-receptor on the surface of cytotoxic T cells. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) different CD8 beta chains (see UniProtKB identifier P10966) and a single CD8 alpha chain (see UniProtKB identifier P01732).

As used herein, the term “CD8 alpha” or “CD8α” refers to one of the two polypeptide chains that make up the CD8 co-receptor (see UniProtKB identifier P01732). The functional CD8 co-receptor is composed of either two CD8α polypeptide chains (i.e., a CD8α homodimer), or a heterodimer of one CD8α polypeptide chain and one CD8β polypeptide chain (i.e., a CD8α/CD8β heterodimer).

As used herein, the term “CD8 beta” or “CD8β” refers the second polypeptide chain of the CD8 glycoprotein (see UniProtKB identifier P10966). The functional CD8 co-receptor may be composed of a heterodimer of one CD8α polypeptide chain and one CD8β polypeptide chain (i.e., a CD8α/CD8β heterodimer).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules and T cell activating therapeutic agents used in the present disclosure are capable of inducing T cell activation. Suitable assays to measure T cell activation are known in the art described herein.

“Immunotherapy” refers to the treatment of a subject afflicted with, or at risk of contracting or suffering a recurrence of, a disease by a method comprising inducing, enhancing, suppressing or otherwise modifying the immune system or an immune response.

As used herein, the term “linked” refers to the association of two or more molecules. The linkage can be covalent or non-covalent. The linkage also can be genetic (i.e., fused). Such linkages can be achieved using a wide variety of art recognized techniques, such as chemical conjugation and recombinant protein production.

“Linker” as used herein refers to a molecule that connect two polypeptide chains. Linker can be a polypeptide linker or a synthetic chemical linker (for example, see disclosed in Protein Engineering, 9(3), 299-305, 1996). The length and sequence of the polypeptide linkers is not particularly limited and can be selected according to the purpose by those skilled in the art. Polypeptide linker comprises one or more amino acids. In some aspects, the polypeptide linker is a peptide with a length of at least 5 amino acids, preferably with a length of 5 to 100, more preferably of 10 to 50 amino acids. In one aspect, said peptide linker is G, S, GS, SG, SGG, GGS, and GSG (with G=glycine and S=serine). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), and bis[2-(succinimidoxycarbonyloxy)ethyl]sulfone (sulfo-BSOCOES).

By “fused” is meant that the components (e.g., an IL-2 molecule and an Fc domain subunit) are linked by peptide bonds, either directly or via one or more peptide linkers.

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, “cancer” refers a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream. Examples of cancer include but are not limited to, carcinoma, lymphoma, blastoma, sarcoma, and leukemia. More particular examples of such cancers include lung cancer, small-cell lung cancer, non-small cell lung (NSCL) cancer, bronchioloalviolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliay cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

The terms “effective amount,” “therapeutically effective amount,” and a “sufficient amount” of, e.g., a cytokine engager or a composition described herein refer to a quantity sufficient to, when administered to a subject including a human effect beneficial or desired results, including alleviation of symptoms; diminishment of the extent of a condition, disorder, or disease; attainment of a stabilized (i.e., not worsening) state of a condition, disorder, or disease; delay in onset or slowing of a condition, disorder, or disease progression; amelioration of a condition, disorder, or disease state or remission (whether partial or total), whether detectable or undetectable; amelioration of at least one measurable physical parameter, not necessarily discernible by a patient; or enhancement or improvement of a condition, disorder, or disease. In some aspects, treatment includes eliciting a clinically significant response without excessive levels of side effects. As such, a “therapeutically effective amount” or synonyms thereof depend on the context in which they are applied. In some aspects, a therapeutically effective amount of an agent (e.g., a cytokine engager or a composition described herein) is an amount that results in a beneficial or desired result in a subject as compared to a control that does not receive the agent.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein “endogenous” refers to any material from or produced inside an organism, cell, tissue or system.

As used herein, the term “exogenous” refers to any material introduced from or produced outside an organism, cell, tissue or system.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In aspects, a control is an identical experiment or identical conditions without administration of a compound (e.g., a compound described herein). In aspects, inhibition of an activity compared to a control is inhibition of an activity by a compound (e.g., as described herein) compared to the activity in the absence of the compound (e.g., as described herein).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some aspects contacting includes allowing a compound described herein to interact with a protein or enzyme.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

As used herein, the term “inhibition”, “inhibit”, “inhibiting” and the like in reference to cell proliferation (e.g., cancer cell proliferation) means negatively affecting (e.g., decreasing proliferation) or killing the cell. In some aspects, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly, an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

The term “pharmaceutical composition” refers to a preparation that is in such form as to permit the biological activity of the active ingredient to be effective, and which contains no additional components which are unacceptably toxic to a subject to which the composition would be administered. Such composition can be sterile, and can comprise a pharmaceutically acceptable carrier, such as physiological saline. Suitable pharmaceutical compositions can comprise one or more of a buffer (e.g., acetate, phosphate or citrate buffer), a surfactant (e.g., polysorbate), a stabilizing agent (e.g., human albumin), a preservative (e.g., benzyl alcohol), and absorption promoter to enhance bioavailability, and/or other conventional solubilizing or dispersing agents.

As used herein, the terms “ug” and “uM” are used interchangeably with “μg” and “μM,” respectively.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Various aspects described herein are described in further detail in the following subsections.

### I. Cytokine Engagers of the Disclosure

The disclosure provides a cytokine engager comprising an antibody that specifically binds a cluster of differentiation 8 (CD8) epitope (also referred to herein as a CD8-binding cytokine engager), wherein the antibody is fused (i.e., linked) to a cytokine (e.g., interleukin-2 (IL-2) or a mutant thereof). A cytokine engager as described herein may also be referred to as a fusion protein and/or immunoconjugate. In some aspects, the disclosure provides a cytokine engager comprising an antibody that specifically binds a CD8 epitope, wherein the antibody is fused to IL-2. In some aspects, the disclosure provides a cytokine engager comprising an antibody that specifically binds a CD8 epitope, wherein the antibody is fused to an IL-2 mutant.

The general benefits of immunoconjugate therapy are readily apparent. For example, an antibody comprised in an immunoconjugate recognizes a tumor-specific epitope and results in targeting of the immunoconjugate molecule to the tumor site. Therefore, high concentrations of IL-2 can be delivered into the tumor microenvironment, thereby resulting in activation and proliferation of a variety of immune effector cells mentioned herein using a much lower dose of the immunoconjugate than would be required for unconjugated IL-2. Moreover, since application of IL-2 in form of immunoconjugates allows lower doses of the cytokine itself, the potential for undesirable side effects of IL-2 is restricted and targeting the IL-2 to a specific site in the body by means of an immunoconjugate may also result in a reduction of systemic exposure and thus less side effects than obtained with unconjugated IL-2. In addition, the increased circulating half-life of an immunoconjugate compared to unconjugated IL-2 contributes to the efficacy of the immunoconjugate. However, this characteristic of IL-2 immunoconjugates may again aggravate potential side effects of the IL-2 molecule: Because of the significantly longer circulating half-life of IL-2 immunoconjugate in the bloodstream relative to unconjugated IL-2, the probability for IL-2 or other portions of the fusion protein molecule to activate components generally present in the vasculature is increased. The same concern applies to other fusion proteins that contain IL-2 fused to another moiety such as Fc or albumin, resulting in an extended half-life of IL-2 in the circulation. Therefore, an immunoconjugate comprising a mutant IL-2 polypeptide as described herein, with reduced toxicity compared to wild type forms of IL-2, is particularly advantageous.

In some aspects, the antibody comprises: a heavy chain complementarity determining region (HCDR) 1, a HCDR2, and a HCDR3, comprising the amino acid sequence of SEQ ID NO: 6, SEQ ID NO: 7, and SEQ ID NO: 8, respectively; and a light chain complementarity determining region (LCDR) 1, a LCDR2, and a LCDR3, comprising the amino acid sequence of SEQ ID NO: 9, SEQ ID NO: 10, and SEQ ID NO: 11, respectively.

In some aspects, the antibody comprises a first heavy chain, a second heavy chain, and two light chains, and wherein: either the first heavy chain or the second heavy chain of the antibody is fused at the C-terminus to the cytokine (e.g., a bivalent cytokine-conjugated antibody wherein each of the first heavy chain and second heavy chain are fused at the C-terminus to a single cytokine molecule, for a total of two cytokine molecules fused to the antibody); or the first heavy chain is fused at the C-terminus to the cytokine and the second heavy chain of the antibody is fused at the C-terminus to a second cytokine (i.e., a bivalent cytokine-conjugated antibody wherein both the cytokine and the second cytokine are fused to the antibody).

The mutant IL-2 polypeptide may be fused to the antibody directly or through a linker peptide, comprising one or more amino acids, typically about 2-20 amino acids. Suitable, non-immunogenic linker peptides include, for example, (G4S)n, (SG4)n, (G4S)n or G4 (SG4)n linker peptides. “n” is generally an integer from 1 to 10, typically from 2 to 4. In one aspect the linker peptide has a length of at least 5 amino acids, in one aspect a length of 5 to 100, in a further aspect of 10 to 50 amino acids. In a particular aspect, the linker peptide has a length of 15 amino acids. In one aspect the linker peptide is (GxS)n or (GXS)nGm with G=glycine, S=serine, and (x=3, n=3, 4, 5 or 6, and m=0, 1, 2 or 3) or (x=4, n=2, 3, 4 or 5 and m=0, 1, 2 or 3), in one aspect x=4 and n=2 or 3, in a further aspect x=4 and n=3. In some aspects, the antibody is fused to the cytokine via a linker comprising SEQ ID NO: 13. In some aspects, the antibody is fused to the cytokine via a linker comprising SEQ ID NO: 14.

In some aspects, the cytokine (i.e., the cytokine fused to the antibody of the cytokine engager) is interleukin-2 (IL-2). In some aspects, the cytokine is an IL-2 mutant (also referred to herein as an IL-2 mutein) comprising one or more of the following substitutions: E61K, E61Q, E61A, E62A, E62K, E62Q, H16A, H16E, H16Q, D20A, D20T, F42A, F42K, R38A, R38E, L72G, L72A, N88R, N88A, C125A, C125S, C125V, Q126A, Q126T. In some aspects, the cytokine is an IL-2 mutant comprising an F42K substitution. In some aspects, the cytokine is an IL-2 mutant comprising an E61K substitution. In some aspects, the first four amino acids of the IL-2 are deleted. In some aspects, one to four amino acids of the IL-2 are deleted. In some aspects, the IL-2 does not comprise SEQ ID NO: 15. In some aspects, the the IL-2 comprises a sequence according to one of SEQ ID NOs: 17-39.

In some aspects, the IL-2 mutant comprises at least one amino acid substitution that reduces the affinity of the IL-2 mutant for an IL-2 receptor compared to a wild type IL-2. In some aspects, the polypeptide comprising an IL-2 mutant provided herein is an agonist of an IL-2R. In some aspects, the IL-2 mutant is a human IL-2 mutant, and the IL-2R is a human IL-2R. In some aspects, the IL-2 mutant binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

IL-2 mutants useful in the disclosure, in addition to having mutations in the region of IL-2 that forms the interface of IL-2 with CD25 or the glycosylation site, also may have one or more mutations in the amino acid sequence outside these regions. Such additional mutations in human IL-2 may provide additional advantages such as increased expression or stability. For example, the cysteine at position 125 may be replaced with a neutral amino acid such as serine, alanine, threonine or valine, yielding C125S IL-2, C125A IL-2, C125T IL-2 or C125V IL-2 respectively, as described in U.S. Pat. No. 4,518,584. As described therein, one may also delete the N-terminal alanine residue of IL-2 yielding such mutants as des-A1 C125S or des-A1 C125A. Alternatively or conjunctively, the IL-2 mutant may include a mutation whereby methionine normally occurring at position 104 of wild type human IL-2 is replaced by a neutral amino acid such as alanine (see U.S. Pat. No. 5,206,344). The resulting mutants, e. g., des-A1 M104A IL-2, des-A1 M104A C125S IL-2, M104A IL-2, M104A C125A IL-2, des-A1 M104A C125A IL-2, or M104A C125S IL-2 (these and other mutants may be found in U.S. Pat. No. 5,116,943 and in Weiger et al., Eur J Biochem 180, 295-300 (1989)) may be used in conjunction with the particular IL-2 mutations of the disclosure.

In some aspects, the IL-2 mutant comprises an additional amino acid mutation at a position corresponding to residue 125 of human IL-2. In one aspect, said additional amino acid mutation is the amino acid substitution C125A.

In some aspects, the antibody (i.e., the antibody of the cytokine engager) comprises a variable heavy chain and a variable light chain that is at least 90%, at least 91% at least 92%, at least 93%, at least 94%, at least 95%, at least 96%, at least 97%, at least 98%, or at least 99% identical to SEQ ID NO: 2 and SEQ ID NO: 1, respectively. In some aspects, the antibody comprises a variable heavy chain and a variable light chain comprising the amino acid sequence of SEQ ID NO: 2 and SEQ ID NO: 1, respectively.

In some aspects, the antibody comprises an Fc region from an IgG antibody. In some aspects, the Fc region is from an IgG1 antibody. In some aspects, the Fc region is derived from a human Fc region.

The Fc domain confers to the immunoconjugate favorable pharmacokinetic properties, including a long serum half-life which contributes to good accumulation in the target tissue and a favorable tissue-blood distribution ratio. At the same time, it may, however, lead to undesirable targeting of the immunoconjugate to cells expressing Fc receptors rather than to the preferred antigen-bearing cells. Moreover, the co-activation of Fc receptor signaling pathways may lead to cytokine release which, in combination with the IL-2 polypeptide and the long half-life of the immunoconjugate, results in excessive activation of cytokine receptors and severe side effects upon systemic administration. In line with this, conventional IgG-IL-2 immunoconjugates have been described to be associated with infusion reactions (see e.g., King et al., J Clin Oncol 22, 4463-4473 (2004)).

In some aspects, the antibody comprises an Fc region comprising the mutations L234F, L235E, and P331S, where the numbering is according to the EU index as in Kabat.

In some aspects, the Fc region comprises mutations M252Y and M428V, herein referred to as “Fc-YV”. In some aspects, the Fc region comprises mutations M252Y and M428L, herein referred to as “Fc-YL”. In some aspects, such mutations enhance binding to FcRn at the acidic pH of the endosome (near 6.5), while losing detectable binding at neutral pH (about 7.2), allowing for enhanced FcRn mediated recycling and extended half-life.

In some aspects, each heavy chain (i.e., a first heavy chain and a second heavy chain) of the antibody comprises SEQ ID NO: 4 and each light chain of the antibody comprises SEQ ID NO: 3. In some aspects, each heavy chain of the antibody comprises SEQ ID NO: 5 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, and wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker).

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, and wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), and wherein the antibody comprises a variable heavy chain and a variable light chain that is at least 90%, at least 91% at least 92%, at least 93%, at least 94%, at least 95%, at least 96%, at least 97%, at least 98%, or at least 99% identical to SEQ ID NO: 2 and SEQ ID NO: 1, respectively. In some aspects, the antibody comprises a variable heavy chain and a variable light chain comprising the amino acid sequence of SEQ ID NO: 2 and SEQ ID NO: 1, respectively.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), and wherein the antibody comprises an Fc region is from an IgG1 antibody.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), and wherein the antibody comprises a variable heavy chain and a variable light chain that is at least 90%, at least 91% at least 92%, at least 93%, at least 94%, at least 95%, at least 96%, at least 97%, at least 98%, or at least 99% identical to SEQ ID NO: 2 and SEQ ID NO: 1, and wherein the antibody comprises an Fc region is from an IgG1 antibody.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), and wherein the antibody comprises an Fc region comprising the mutations L234F, L235E, and P331S, where the numbering is according to the EU index as in Kabat.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), wherein the antibody comprises an Fc region comprising the mutations L234F, L235E, and P331S, where the numbering is according to the EU index as in Kabat, and wherein each heavy chain of the antibody comprises SEQ ID NO: 4 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), wherein the antibody comprises a variable heavy chain and a variable light chain comprising the amino acid sequence of SEQ ID NO: 2 and SEQ ID NO: 1, respectively, wherein the antibody comprises an Fc region comprising the mutations L234F, L235E, and P331S, where the numbering is according to the EU index as in Kabat, and wherein each heavy chain of the antibody comprises SEQ ID NO: 4 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), wherein the antibody comprises an Fc region comprising the mutations L234F, L235E, and P331S, where the numbering is according to the EU index as in Kabat, and wherein each heavy chain of the antibody comprises SEQ ID NO: 5 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an E61K substitution (e.g., the antibody is fused to a first IL-2 comprising an E61K substitution and a second IL-2 comprising an E61K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an E61K substitution via a first linker and a second IL-2 comprising an E61K substitution via a second linker), wherein the antibody comprises a variable heavy chain and a variable light chain comprising the amino acid sequence of SEQ ID NO: 2 and SEQ ID NO: 1, respectively, wherein the antibody comprises an Fc region comprising the mutations L234F, L235E, and P331S, where the numbering is according to the EU index as in Kabat, and wherein each heavy chain of the antibody comprises SEQ ID NO: 5 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, and wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker).

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, and wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), and wherein the antibody comprises a variable heavy chain and a variable light chain that is at least 90%, at least 91% at least 92%, at least 93%, at least 94%, at least 95%, at least 96%, at least 97%, at least 98%, or at least 99% identical to SEQ ID NO: 2 and SEQ ID NO: 1, respectively. In some aspects, the antibody comprises a variable heavy chain and a variable light chain comprising the amino acid sequence of SEQ ID NO: 2 and SEQ ID NO: 1, respectively.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), and wherein the antibody comprises an Fc region is from an IgG1 antibody.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), and wherein the antibody comprises a variable heavy chain and a variable light chain that is at least 90%, at least 91% at least 92%, at least 93%, at least 94%, at least 95%, at least 96%, at least 97%, at least 98%, or at least 99% identical to SEQ ID NO: 2 and SEQ ID NO: 1, and wherein the antibody comprises an Fc region is from an IgG1 antibody.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), and wherein the antibody comprises an Fc region comprising the mutations L234F, L235E, and P331S, where the numbering is according to the EU index as in Kabat.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), wherein the antibody comprises an Fc region comprising the mutations L234F, L235E, and P331S, where the numbering is according to the EU index as in Kabat, and wherein each heavy chain of the antibody comprises SEQ ID NO: 4 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), wherein the antibody comprises a variable heavy chain and a variable light chain comprising the amino acid sequence of SEQ ID NO: 2 and SEQ ID NO: 1, respectively, wherein the antibody comprises an Fc region comprising the mutations L234F, L235E, and P331S, where the numbering is according to the EU index as in Kabat, and wherein each heavy chain of the antibody comprises SEQ ID NO: 4 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), wherein the antibody comprises an Fc region comprising the mutations L234F, L235E, and P331S, where the numbering is according to the EU index as in Kabat, and wherein each heavy chain of the antibody comprises SEQ ID NO: 5 and each light chain of the antibody comprises SEQ ID NO: 3.

In some aspects, the disclosure provides a cytokine engager comprising an antibody that specifically binds a CD8 epitope, wherein the antibody is fused to an IL-2 comprising an F42K substitution (e.g., the antibody is fused to a first IL-2 comprising an F42K substitution and a second IL-2 comprising an F42K substitution), wherein the antibody comprises: a HCDR1, a HCDR2, and a HCDR3, comprising the amino acid sequence of SEQ ID NO: 6, SEQ ID NO: 7, and SEQ ID NO: 8, respectively; and a LCDR1, a LCDR2, and a LCDR3, comprising the amino acid sequence of SEQ ID NO: 9, SEQ ID NO: 10, and SEQ ID NO: 11, respectively, wherein the antibody is fused to the cytokine via a linker comprising SEQ ID NO: 14 (e.g., the antibody is fused to a first IL-2 comprising an F42K substitution via a first linker and a second IL-2 comprising an F42K substitution via a second linker), wherein the antibody comprises a variable heavy chain and a variable light chain comprising the amino acid sequence of SEQ ID NO: 2 and SEQ ID NO: 1, respectively, wherein the antibody comprises an Fc region comprising the mutations L234F, L235E, and P331S, where the numbering is according to the EU index as in Kabat, and wherein each heavy chain of the antibody comprises SEQ ID NO: 5 and each light chain of the antibody comprises SEQ ID NO: 3.

In some aspects, the cytokine engager binds to CD8⁺ T cells and promotes proliferation of the CD8⁺ T cells. In some aspects, wherein the CD8⁺ T cells are cytotoxic CD8⁺ T cells.

In some aspects, the cytokine engager binds to CD8⁺ T cells and promotes proliferation of the CD8⁺ T cells relative to CD8⁺ T cell proliferation in the absence of the cytokine engager. In some aspects, the cytokine engager binds to CD8⁺ T cells and promotes proliferation of the CD8⁺ T cells by at least 1.5-fold, at least 2-fold, at least 3-fold, or by at least 5-fold relative to CD8⁺ T cell proliferation in the absence of the cytokine engager. In some aspects, the cytokine engager binds to CD8⁺ T cells and promotes proliferation of the CD8⁺ T cells by about 1.5-fold relative to CD8⁺ T cell proliferation in the absence of the cytokine engager. In some aspects, the cytokine engager binds to CD8⁺ T cells and promotes proliferation of the CD8⁺ T cells by about 2-fold relative to CD8⁺ T cell proliferation in the absence of the cytokine engager. In some aspects, the cytokine engager binds to CD8⁺ T cells and promotes proliferation of the CD8⁺ T cells by about 3-fold relative to CD8⁺ T cell proliferation in the absence of the cytokine engager. In some aspects, the cytokine engager binds to CD8⁺ T cells and promotes proliferation of the CD8⁺ T cells by about 5-fold relative to CD8⁺ T cell proliferation in the absence of the cytokine engager. In some aspects, the cytokine engager binds to CD8⁺ T cells and promotes proliferation of the CD8⁺ T cells by greater than 5-fold relative to CD8⁺ T cell proliferation in the absence of the cytokine engager.

In some aspects, the cytokine engager increases pSTAT5 expression in CD8⁺ T cells relative to pSTAT5 expression in the absence of the cytokine engager. In some aspects, the cytokine engager increases pSTAT5 expression in CD8⁺ T cells by at least 1.5-fold, at least 2-fold, at least 3-fold, or by at least 5-fold relative to pSTAT5 expression in the absence of the cytokine engager. In some aspects, the cytokine engager increases pSTAT5 expression in CD8⁺ T cells by about 1.5-fold relative to pSTAT5 expression in the absence of the cytokine engager. In some aspects, the cytokine engager increases pSTAT5 expression in CD8⁺ T cells by about 2-fold relative to pSTAT5 expression in the absence of the cytokine engager. In some aspects, the cytokine engager increases pSTAT5 expression in CD8⁺ T cells by about 3-fold relative to pSTAT5 expression in the absence of the cytokine engager. In some aspects, the cytokine engager increases pSTAT5 expression in CD8⁺ T cells by about 5-fold relative to pSTAT5 expression in the absence of the cytokine engager. In some aspects, the cytokine engager increases pSTAT5 expression in CD8⁺ T cells by greater than 5-fold relative to pSTAT5 expression in the absence of the cytokine engager.

In some aspects, the cytokine engager promotes proliferation of the CD8⁺ T cells relative to NK cell proliferation in the presence of the cytokine engager. In some aspects, the cytokine engager promotes proliferation of the CD8⁺ T cells by at least 1.5-fold, at least 2-fold, at least 3-fold, or by at least 5-fold relative to NK cell proliferation in the presence of the cytokine engager. In some aspects, the cytokine engager binds to CD8⁺ T cells and promotes proliferation of the CD8⁺ T cells by at about 1.5-fold relative to NK cell proliferation in the presence of the cytokine engager. In some aspects, the cytokine engager binds to CD8⁺ T cells and promotes proliferation of the CD8⁺ T cells by at about 2-fold relative to NK cell proliferation in the presence of the cytokine engager. In some aspects, the cytokine engager binds to CD8⁺ T cells and promotes proliferation of the CD8⁺ T cells by at about 3-fold relative to NK cell proliferation in the presence of the cytokine engager. In some aspects, the cytokine engager binds to CD8⁺ T cells and promotes proliferation of the CD8⁺ T cells by at about 5-fold relative to NK cell proliferation in the presence of the cytokine engager. In some aspects, the cytokine engager binds to CD8⁺ T cells and promotes proliferation of the CD8⁺ T cells by greater than 5-fold relative to NK cell proliferation in the presence of the cytokine engager.

In some aspects, the cytokine engager promotes the expression of CD69 of CD8⁺ T cells. In some aspects, the cytokine engager induces the expression of CD69 in CD8⁺ T cells by at least 1.5-fold, at least 2-fold, at least 3-fold, or by at least 5-fold relative to CD69 expression in NK cells in the presence of the cytokine engager. In some aspects, the cytokine engager induces the expression of CD69 in CD8⁺ T cells by about 1.5-fold relative to CD69 expression in NK cells in the presence of the cytokine engager. In some aspects, the cytokine engager induces the expression of CD69 in CD8⁺ T cells by about 2-fold relative to CD69 expression in NK cells in the presence of the cytokine engager. In some aspects, the cytokine engager induces the expression of CD69 in CD8⁺ T cells by about 3-fold relative to CD69 expression in NK cells in the presence of the cytokine engager. In some aspects, the cytokine engager induces the expression of CD69 in CD8⁺ T cells by about 5-fold relative to CD69 expression in NK cells in the presence of the cytokine engager. In some aspects, the cytokine engager induces the expression of CD69 in CD8⁺ T cells by greater than 5-fold relative to CD69 expression in NK cells in the presence of the cytokine engager.

In some aspects, the cytokine engager reduces or attenuates the suppressive activity of regulatory T cells (Tregs) on CD8⁺ T cells. In some aspects, the cytokine engager reduces the suppressive activity of Tregs by on CD8⁺ T cells by at least 10%, at least 20%, at least 30%, or by at least 50%. In some aspects, the cytokine engager reduces the suppressive activity of Tregs by on CD8⁺ T cells by about 10%. In some aspects, the cytokine engager reduces the suppressive activity of Tregs by on CD8⁺ T cells by about 20%. In some aspects, the cytokine engager reduces the suppressive activity of Tregs by on CD8⁺ T cells by about 30%. In some aspects, the cytokine engager reduces the suppressive activity of Tregs by on CD8⁺ T cells by about 50%. In some aspects, the cytokine engager reduces the suppressive activity of Tregs by on CD8⁺ T cells by greater than 50%.

In some aspects, the cytokine engager (e.g., the CD8-binding cytokine engager) enhances anti-tumor T cell responses or natural killer cell responses while avoiding Tregs, peripheral T cells, and endothelial cells. In some aspects, the cytokine engager targets the IL-2 mutant to activated T cells. In some aspects, the IL-2 mutant binds and modulates an IL-2R only when the IL-2R is on the same cell as the CD8 (i.e., the CD8 epitope) bound by the cytokine engager. In some aspects, the IL-2 mutant does not bind or activate an IL-2R when the IL-2R is on a different cell than the cell expressing the CD8 (i.e., the CD8 epitope) bound by cytokine engager.

### II. Nucleic Acids, Vectors, Host Cells, and Pharmaceutical Compositions of the Disclosure

The disclosure provides an isolated nucleic acid sequence (i.e., a polynucleotide) encoding a cytokine engager (e.g., a CD8-binding cytokine engager) as described herein. The disclosure further provides a pharmaceutical composition comprising the cytokine engager as described herein.

In some aspects, the isolated nucleic acid sequence of the present disclosure is present in a vector. As such, provided herein are vectors comprising an isolated nucleic acid sequence of the present disclosure. In some aspects, the present disclosure is directed to a vector comprising an isolated nucleic acid sequence encoding a cytokine engager (e.g., a CD8-binding cytokine engager), as described herein. In other aspects, the present disclosure is directed to a vector comprising an isolated nucleic acid sequence encoding a cytokine engager (e.g., a CD8-binding cytokine engager) comprising an antibody fused to a cytokine (e.g., IL-2), as disclosed herein.

Any vector known in the art can be suitable for the present disclosure. In some aspects, the vector is a viral vector. In some aspects, the vector is a retroviral vector, a DNA vector, a murine leukemia virus vector, an SFG vector, a plasmid, an RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

In other aspects, provided herein are host cells comprising an isolated nucleic acid sequence or a vector of the present disclosure. In some aspects, the present disclosure is directed to host cells, e.g., in vitro cells, comprising an isolated nucleic acid sequence encoding a CD8-binding cytokine engager, as described herein. In some aspects, the present disclosure is directed to host cells, e.g., in vitro cells, comprising an isolated nucleic acid sequence encoding a cytokine engager that specifically binds to CD8 (i.e., a CD8-binding cytokine engager), as disclosed herein. In other aspects, the present disclosure is directed to in vitro cells comprising a polypeptide encoded by an isolated nucleic acid sequence encoding a cytokine engager that specifically binds to CD8.

In a further aspect, a host cell comprising one or more nucleic acid sequences of the disclosure is provided. In some aspects, a host cell comprising one or more vectors of the disclosure is provided. The nucleic acid sequences and vectors may incorporate any of the features, singly or in combination, described herein in relation to nucleic acid sequences and vectors, respectively. In one such aspect a host cell comprises (e.g., has been transformed or transfected with) one or more vector comprising one or more nucleic acid sequences that encodes the cytokine engager of the disclosure. As used herein, the term “host cell” refers to any kind of cellular system which can be engineered to generate the cytokine engagers of the disclosure or fragments thereof. Host cells are suitable for replicating and for supporting expression of cytokine engagers. Such cells may be transfected or transduced as appropriate with the particular expression vector and large quantities of vector containing cells can be grown for seeding large scale fermenters to obtain sufficient quantities of the cytokine engager for clinical applications.

Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one aspect, the host cell is a eukaryotic cell, preferably a mammalian cell, such as a Chinese Hamster Ovary (CHO) cell, a human embryonic kidney (HEK) cell or a lymphoid cell (e.g., YO, NSO, Sp20 cell).

Any cell may be used as a host cell for the isolated nucleic acid sequences, the vectors, or the polypeptides of the present disclosure. In some aspects, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some aspects, the cell is a human cell.

In one aspect, a method of producing a cytokine engager according to the disclosure is provided, wherein the method comprises culturing a host cell comprising one or more nucleic acid sequence encoding the cytokine engager, as provided herein, under conditions suitable for expression of the cytokine engager, and optionally recovering the cytokine engager from the host cell (or host cell culture medium).

Other aspects of the present disclosure are directed to compositions comprising an isolated nucleic acid sequence described herein, a vector described herein, a polypeptide described herein, or cell described herein. In some aspects, the composition comprises a pharmaceutically acceptable carrier, diluent, solubilizer, emulsifier, preservative and/or adjuvant. In some aspects, the composition comprises an excipient. In one aspect, the composition comprises an isolated nucleic acid sequence encoding a cytokine engager, wherein the cytokine engager comprises an antibody that specifically binds to CD8, wherein the antibody is fused to a cytokine (e.g., IL-2). In another aspect, the composition comprises a cytokine engager encoded by an isolated nucleic acid sequence of the present disclosure, wherein the cytokine engager comprises an antibody that specifically binds to CD8, wherein the antibody is fused to a cytokine (e.g., IL-2). In another aspect, the composition comprises a cell comprising an isolated nucleic acid sequence encoding a cytokine engager, wherein the cytokine engager comprises an antibody that specifically binds to CD8, wherein the antibody is fused to a cytokine (e.g., IL-2). In another aspect, the composition comprises a cytokine engager as described herein, wherein the cytokine engager comprises an antibody that specifically binds to CD8, wherein the antibody is fused to a cytokine (e.g., IL-2).

The disclosure also provides pharmaceutical compositions comprising CD8-binding cytokine engager and a pharmaceutically acceptable carrier. The pharmaceutical compositions comprising a CD8-binding cytokine engager are for use in, but not limited to, diagnosing, detecting, or monitoring a disorder, in preventing, treating, managing, or ameliorating of a disorder or one or more symptoms thereof, and/or in research. In an aspect, provided herein is a pharmaceutical composition comprising a CD8-binding cytokine engager as described herein and a pharmaceutically acceptable carrier. In an aspect, provided herein is a pharmaceutical composition comprising a CD8-binding cytokine engager as described herein, wherein the cytokine engager comprises an antibody that specifically binds to CD8, wherein the antibody is fused to a cytokine, and a pharmaceutically acceptable carrier.

In some aspects, the cytokine is interleukin-2 (IL-2). In some aspects, the cytokine is an IL-2 mutant comprising one or more of the following substitutions: E61K, E61Q, E61A, E62A, E62K, E62Q, H16A, H16E, H16Q, D20A, D20T, F42A, F42K, R38A, R38E, L72G, L72A, N88R, N88A, C125A, C125S, C125V, Q126A, or Q126T. In some aspects, the cytokine is an IL-2 comprising a F42K substitution. In some aspects, the cytokine is an IL-2 comprising an E61K substitution. In some aspects, the first four amino acids of the IL-2 are deleted. In some aspects, the IL-2 does not comprise SEQ ID NO: 15. In some aspects, the IL-2 comprises a sequence according to one of SEQ ID NOs: 17-39.

In various aspects, kits for use in the laboratory and therapeutic applications described herein are within the scope of the present disclosure. Such kits may comprise a carrier, package, or container that is compartmentalized to receive one or more containers such as vials, tubes, and the like, each of the container(s) comprising one of the separate elements to be used in a method disclosed herein, along with a label or insert comprising instructions for use, such as a use described herein. Kits may comprise a container comprising a drug moiety. The present disclosure also provides a CD8-binding cytokine engager, or pharmaceutical compositions thereof, packaged in a hermetically sealed container, such as an ampoule or sachette, indicating the quantity of the agent.

Kits may comprise the container described above, and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label may be present on or with the container to indicate that the composition is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that the composition is used for diagnosing or treating a condition, such as a cancer a described herein.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the disclosure are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

Sequences.

| SEQ | | |
|---|---|---|
| ID | Description | Sequences |
| 1 | Variable | DIQMTQSPSSLSASVGDRVTITCKGSQDISNYLAWYQQKPGK |
| | light | APKLLIYNTDILHTGVPSRFSGSGSGTDFTFTISSLQPEDIA |
| | chain | TYYCYQYDTGYTFGQGTKVEIK |
| 2 | variable | QVQLVESGGGVVQPGRSLRLSCAASGFTFSDFGMNWVRQAPG |
| | heavy | KGLEWVALIYYDGSNKFYADSVKGRFTISRDNSKNTLYLQMN |
| | chain | SLRAEDTAVYYCAKPHYDGYYHFFDSWGQGTLVTVSS |
| 3 | full | DIQMTQSPSSLSASVGDRVTITCKGSQDISNYLAWYQQKPGK |
| | light | APKLLIYNTDILHTGVPSRFSGSGSGTDFTFTISSLQPEDIA |
| | Chain | TYYCYQYDTGYTFGQGTKVEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 4 | full | QVQLVESGGGVVQPGRSLRLSCAASGFTFSDFGMNWVRQAPG |
| | heavy | KGLEWVALIYYDGSNKFYADSVKGRFTISRDNSKNTLYLQMN |
| | chain | SLRAEDTAVYYCAKPHYDGYYHFFDSWGQGTLVTVSSASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKRVEPKSCDKTHTCPPCPAPEFEGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPASI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 5 | Heavy | QVQLVESGGGVVQPGRSLRLSCAASGFTFSDFGMNWVRQAPG |
| | chain + | KGLEWVALIYYDGSNKFYADSVKGRFTISRDNSKNTLYLQMN |
| | linker + | SLRAEDTAVYYCAKPHYDGYYHFFDSWGQGTLVTVSSASTKG |
| | IL2 | PSVFPLAPSSKSTSGGTAALGCLVKDYFPEPVTVSWNSGALT |
| | (E61K) | SGVHTFPAVLQSSGLYSLSSVVTVPSSSLGTQTYICNVNHKP |
| | | SNTKVDKRVEPKSCDKTHTCPPCPAPEFEGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPASI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGKGGGGSGGGGSS |
| | | TKKTQLQLEHLLLDLQMILNGINNYKNPKLTRMLTFKFYMPK |
| | | KATELKHLQCLEKELKPLEEVLNLAQSKNFHLRPRDLISNIN |
| | | VIVLELKGSETTFMCEYADETATIVEFLNRWITFCQSIISTL |
| | | T |
| 6 | HCDR1 | DFGMN |
| 7 | HCDR2 | LIYYDGSNKFYADSVKG |
| 8 | HCDR3 | PHYDGYYHFFDS |
| 9 | LCDR1 | KGSQDISNYLA |
| 10 | LCDR2 | NTDILHT |
| 11 | LCDR3 | YQYDTGYT |
| 12 | Mutant | SSTKKTQLQLEHLLLDLQMILNGINNYKNPKLTRMLTFKFYM |
| | IL2 | PKKATELKHLQCLEKELKPLEEVLNLAQSKNFHLRPRDLISN |
| | (E61K) | INVIVLELKGSETTFMCEYADETATIVEFLNRWITFCQSIIS |
| | | TLT |
| 13 | Single | GGGGS |
| | linker | |
| 14 | Linker | GGGGSGGGG |
| | (2XG4S) | |
| 15 | IL2 | APTS |
| | residues | |
| | removed | |
| 16 | WT IL2 | APTSSSTKKTQLQLEHLLLDLQMILNGINNYKNPKLTRMLTF |
| | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLT |
| 17 | Mutant | SSTKKTQLQLEHLLLDLQMILNGINNYKNPKLTRMLTFKFYM |
| | IL2 | PKKATELKHLQCLEQELKPLEEVLNLAQSKNFHLRPRDLISN |
| | (E61Q) | INVIVLELKGSETTFMCEYADETATIVEFLNRWITFCQSIIS |
| | | TLT |
| 18 | Mutant | SSTKKTQLQLEHLLLDLQMILNGINNYKNPKLTRMLTFKFYM |
| | IL2 | PKKATELKHLQCLEAELKPLEEVLNLAQSKNFHLRPRDLISN |
| | (E61A) | INVIVLELKGSETTFMCEYADETATIVEFLNRWITFCQSIIS |
| | | TLT |
| 19 | Mutant | SSTKKTQLQLEHLLLDLQMILNGINNYKNPKLTRMLTFKFYM |
| | IL2 | PKKATELKHLQCLEEALKPLEEVLNLAQSKNFHLRPRDLISN |
| | (E62A) | INVIVLELKGSETTFMCEYADETATIVEFLNRWITFCQSIIS |
| | | TLT |
| 20 | Mutant | SSTKKTQLQLEHLLLDLQMILNGINNYKNPKLTRMLTFKFYM |
| | IL2 | PKKATELKHLQCLEEKLKPLEEVLNLAQSKNFHLRPRDLISN |
| | (E62K) | INVIVLELKGSETTFMCEYADETATIVEFLNRWITFCQSIIS |
| | | TLT |
| 21 | Mutant | SSTKKTQLQLEHLLLDLQMILNGINNYKNPKLTRMLTFKFYM |
| | IL2 | PKKATELKHLQCLEEQLKPLEEVLNLAQSKNFHLRPRDLISN |
| | (E62Q) | INVIVLELKGSETTFMCEYADETATIVEFLNRWITFCQSIIS |
| | | TLT |
| 22 | Mutant | SSTKKTQLQLEALLLDLQMILNGINNYKNPKLTRMLTFKFYM |
| | IL2 | PKKATELKHLQCLEEELKPLEEVLNLAQSKNFHLRPRDLISN |
| | (H16A) | INVIVLELKGSETTFMCEYADETATIVEFLNRWITFCQSIIS |
| | | TLT |
| 23 | Mutant | SSTKKTQLQLEELLLDLQMILNGINNYKNPKLTRMLTFKFYM |
| | IL2 | PKKATELKHLQCLEEELKPLEEVLNLAQSKNFHLRPRDLISN |
| | (H16E) | INVIVLELKGSETTFMCEYADETATIVEFLNRWITFCQSIIS |
| | | TLT |
| 24 | Mutant | SSTKKTQLQLEQLLLDLQMILNGINNYKNPKLTRMLTFKFYM |
| | IL2 | PKKATELKHLQCLEEELKPLEEVLNLAQSKNFHLRPRDLISN |
| | (H16Q) | INVIVLELKGSETTFMCEYADETATIVEFLNRWITFCQSIIS |
| | | TLT |
| 25 | Mutant | SSTKKTQLQLEHLLLALQMILNGINNYKNPKLTRMLTFKFYM |
| | IL2 | PKKATELKHLQCLEEELKPLEEVLNLAQSKNFHLRPRDLISN |
| | (D20A) | INVIVLELKGSETTFMCEYADETATIVEFLNRWITFCQSIIS |
| | | TLT |
| 26 | Mutant | SSTKKTQLQLEHLLLTLQMILNGINNYKNPKLTRMLTFKFYM |
| | IL2 | PKKATELKHLQCLEEELKPLEEVLNLAQSKNFHLRPRDLISN |
| | (D20T) | INVIVLELKGSETTFMCEYADETATIVEFLNRWITFCQSIIS |
| | | TLT |
| 27 | Mutant | SSTKKTQLQLEHLLLDLQMILNGINNYKNPKLTRMLTAKFYM |
| | IL2 | PKKATELKHLQCLEEELKPLEEVLNLAQSKNFHLRPRDLISN |
| | (F42A) | INVIVLELKGSETTFMCEYADETATIVEFLNRWITFCQSIIS |
| | | TLT |
| 28 | Mutant | SSTKKTQLQLEHLLLDLQMILNGINNYKNPKLTRMLTKKFYM |
| | IL2 | PKKATELKHLQCLEEELKPLEEVLNLAQSKNFHLRPRDLISN |
| | (F42K) | INVIVLELKGSETTFMCEYADETATIVEFLNRWITFCQSIIS |
| | | TLT |
| 29 | Mutant | SSTKKTQLQLEHLLLDLQMILNGINNYKNPKLTAMLTFKFYM |
| | IL2 | PKKATELKHLQCLEEELKPLEEVLNLAQSKNFHLRPRDLISN |
| | (R38A) | INVIVLELKGSETTFMCEYADETATIVEFLNRWITFCQSIIS |
| | | TLT |
| 30 | Mutant | SSTKKTQLQLEHLLLDLQMILNGINNYKNPKLTEMLTFKFYM |
| | IL2 | PKKATELKHLQCLEEELKPLEEVLNLAQSKNFHLRPRDLISN |
| | (R38E) | INVIVLELKGSETTFMCEYADETATIVEFLNRWITFCQSIIS |
| | | TLT |
| 31 | Mutant | SSTKKTQLQLEHLLLDLQMILNGINNYKNPKLTRMLTFKFYM |
| | IL2 | PKKATELKHLQCLEEELKPLEEVLNGAQSKNFHLRPRDLISN |
| | (L72G) | INVIVLELKGSETTFMCEYADETATIVEFLNRWITFCQSIIS |
| | | TLT |
| 32 | Mutant | SSTKKTQLQLEHLLLDLQMILNGINNYKNPKLTRMLTFKFYM |
| | IL2 | PKKATELKHLQCLEEELKPLEEVLNAAQSKNFHLRPRDLISN |
| | (L72A) | INVIVLELKGSETTFMCEYADETATIVEFLNRWITFCQSIIS |
| | | TLT |
| 33 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISR |
| | (N88R) | INVIVLELKGSETTFMCEYADETATIVEFLNRWITFCQSIIS |
| | | TLT |
| 34 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISA |
| | (N88A) | INVIVLELKGSETTFMCEYADETATIVEFLNRWITFCQSIIS |
| | | TLT |
| 35 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISN |
| | (C125A) | INVIVLELKGSETTFMCEYADETATIVEFLNRWITFAQSIIS |
| | | TLT |
| 36 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISN |
| | (C125S) | INVIVLELKGSETTFMCEYADETATIVEFLNRWITFSQSIIS |
| | | TLT |
| 37 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISN |
| | (C125V) | INVIVLELKGSETTFMCEYADETATIVEFLNRWITFVQSIIS |
| | | TLT |
| 38 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISN |
| | (Q126A) | INVIVLELKGSETTFMCEYADETATIVEFLNRWITFCASIIS |
| | | TLT |
| 39 | Mutant | SSTKKTQLQLEHLLLDLQMILNGINNYKNPKLTRMLTFKFYM |
| | IL2 | PKKATELKHLQCLEEELKPLEEVLNLAQSKNFHLRPRDLISN |
| | (Q126T) | INVIVLELKGSETTFMCEYADETATIVEFLNRWITFCTSIIS |
| | | TLT |

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### III. Methods of the Disclosure

The cytokine engagers described herein may be used for therapeutic applications, such as expanding T cells (e.g., CD8⁺ T cells), stimulating the immune system, and in the treatment of cancer.

In some aspects, a cytokine engager as described herein or a pharmaceutical composition as described herein may be used in a method of expanding T cells, wherein the cytokine engager or pharmaceutical composition is contacted to a population of T cells.

In some aspects, the cytokine engager is administered to a subject in need comprising the population of T cells. In some aspects, the population of T cells is endogenous to the subject. In some aspects, the subject is a cancer patient.

In some aspects, the CD8 T cells preferentially expand compared to CD4 T cells. In some aspects, the CD8 T cells preferentially expand compared to NK cells. In some aspects, the CD8 T cells preferentially expand compared to CD4 T cells, wherein the expansion is compared to contacting SEQ ID NO: 12 to a population of T cells.

In some aspects, a cytokine engager as described herein or a pharmaceutical composition as described herein may be used in a method of treatment of the human or animal body. Related aspects of the disclosure provide:
(i) a cytokine engager as described herein or a composition as described herein for use as a medicament;
(ii) a cytokine engager as described herein or a composition as described herein for use in a method of treatment of a disease or disorder;
(iii) a method for inhibiting the growth of a tumor cell, wherein the method comprises contacting the tumor cell with a cytokine engager as described herein or a composition as described herein;
(iv) a method for stimulating the immune system in a subject, wherein the method comprises administering to the subject in need thereof a therapeutically effective amount of a cytokine engager as described herein or a composition as described herein.
(v) a cytokine engager as described herein or a composition as described herein in the manufacture of a medicament for use in the treatment of a disease or disorder; and,
(vi) a method of treating a disease or disorder in a subject, wherein the method comprises administering to the subject in need thereof a therapeutically effective amount of a cytokine engager as described herein or a composition as described herein.

The subject may be a patient, preferably a human patient, e.g., a patient suffering from cancer.

In a further aspect, the disclosure provides for the use of a cytokine engager of the disclosure in the manufacture or preparation of a medicament. In one aspect, the medicament is for the treatment of a disease in an individual in need thereof. In one aspect, the medicament is for use in a method of treating a disease comprising administering to an individual having the disease a therapeutically effective amount of the medicament. In certain aspects the disease to be treated is a proliferative disorder. In a particular aspect the disease is cancer. In one aspect, the method further comprises administering to the individual a therapeutically effective amount of at least one additional therapeutic agent, e.g., an anti-cancer agent if the disease to be treated is cancer. In a further aspect, the medicament is for stimulating the immune system. In a further aspect, the medicament is for use in a method of stimulating the immune system in an individual comprising administering to the individual an effective amount of the medicament to stimulate the immune system.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment. In some aspects, the method is a method of treating cancer.

Treatment as a prophylactic measure (i.e., prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

Some aspects of the present disclosure are directed to a method for inhibiting the growth of a tumor cell comprising contacting said tumor cell with a cytokine engager as described herein (e.g., a CD8-binding cytokine engager) Some aspects of the present disclosure are directed to a method for inhibiting the growth of a tumor cell comprising contacting said tumor cell with: a cytokine engager comprising an antibody that specifically binds a cluster of differentiation 8 (CD8), wherein the antibody is fused to a cytokine. In some aspects, the antibody comprises: a. a heavy chain complementarity determining region (HCDR) 1, HCDR2, a HCDR3, comprising the amino acid sequence of SEQ ID NO: 6, SEQ ID NO: 7, and SEQ ID NO: 8, respectively; and b. a light chain complementarity determining region (LCDR) 1, LCDR2, a LCDR3, comprising the amino acid sequence of SEQ ID NO: 9, SEQ ID NO: 10, and SEQ ID NO: 11, respectively.

Some aspects of the present disclosure are direct to a method for treating a cancer comprising tumor cells in a subject comprising administering to said subject in need thereof a therapeutically effective amount of a cytokine engager as described herein (e.g., a CD8-binding cytokine engager). Some aspects of the present disclosure are direct to a method for treating a cancer comprising tumor cells in a subject comprising administering to said subject in need thereof a therapeutically effective amount of: a cytokine engager comprising an antibody that specifically binds a cluster of differentiation 8 (CD8), wherein the antibody is fused to a cytokine. In some aspects, the antibody comprises: a. a heavy chain complementarity determining region (HCDR)1, HCDR2, a HCDR3, comprising the amino acid sequence of SEQ ID NO: 6, SEQ ID NO: 7, and SEQ ID NO: 8, respectively; and b. a light chain complementarity determining region (LCDR)1, LCDR2, a LCDR3, comprising the amino acid sequence of SEQ ID NO: 9, SEQ ID NO: 10, and SEQ ID NO: 11, respectively.

Nonlimiting exemplary cancers that may be treated with modified IL-2 containing polypeptides provided herein include basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia. In some aspects, the cancer is melanoma, renal cell carcinoma, gastric, or non-small cell lung cancer.

In some aspects, the cytokine engager administered in the form of a pharmaceutical composition, which may comprise at least one component in addition to the cytokine engager. Another aspect of the disclosure therefore provides a pharmaceutical composition comprising a cytokine engager as described herein. Further chemical modification of the cytokine engager of the disclosure may be desirable. For example, problems of immunogenicity and short half-life may be improved by conjugation to substantially straight chain polymers such as polyethylene glycol (PEG) or polypropylene glycol (PPG) (see e.g., WO 87/00056).

Certain aspects of the present disclosure are directed to methods of treating a disease or condition in a subject in need thereof, comprising administering to the subject a composition disclosed herein. In some aspects, the disease or condition comprises a cancer. In some aspects, the cancer is locally progressed. In some aspects, the cancer is metastatic. In some aspects, the cancer is recurrent. In some aspects, the cancer is relapsed.

The compositions disclosed herein, e.g., a cytokine engager disclosed herein, can be used in combination with other anti-cancer therapies, including one or more additional immunotherapies. In some aspects, the compositions disclosed herein are administered concurrently with the additional anti-cancer agent. In some aspects, the compositions disclosed herein and the additional anti-cancer agent are administered sequentially (e.g., on the same day or on different days).

The in vivo effect of a disclosed cytokine engager therapeutic composition can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Patient-derived xenografts (PDX), in which tumor tissues from patients are implanted into immunocompromised or humanized mice (see, e.g., Liu Y et al. Signal Transduct. Target Ther. 2023; 8(1):160), may also be used to evaluate the in vivo effect of a disclosed CD8-binding cytokine engager therapeutic composition.

Apoptosis may also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al., eds. (1992) pp. 3.17.1-3.17.16). Cells also can be labeled with a DNA dye (e.g., acridine orange, ethidium bromide, or propidium iodide) and the cells observed for chromatin condensation and margination along the inner nuclear membrane. Apoptosis may also be determined, in some aspects, by screening for caspase activity. In some aspects, a Caspase-Glo® Assay can be used to measure activity of caspase-3 and caspase-7. In some aspects, the assay provides a luminogenic caspase-3/7 substrate in a reagent optimized for caspase activity, luciferase activity, and cell lysis. In some aspects, adding Caspase-Glo® 3/7 Reagent in an “add-mix-measure” format may result in cell lysis, followed by caspase cleavage of the substrate and generation of a “glow-type” luminescent signal, produced by luciferase. In some aspects, luminescence may be proportional to the amount of caspase activity present and can serve as an indicator of apoptosis. Other morphological changes that can be measured to determine apoptosis include, e.g., cytoplasmic condensation, increased membrane blebbing, and cellular shrinkage. Determination of any of these effects on cancer cells indicates that an ADC is useful in the treatment of cancers.

Cell viability may be measured, e.g., by determining in a cell the uptake of a dye such as neutral red, trypan blue, Crystal Violet, or ALAMAR™ blue (see, e.g., Page et al. (1993) Intl J Oncology 3:473-6). In such an assay, the cells are incubated in media containing the dye, the cells are washed, and the remaining dye, reflecting cellular uptake of the dye, is measured spectrophotometrically. Cell viability may also be measured, e.g., by quantifying ATP, an indicator of metabolically active cells. In certain aspects, in vitro potency and/or cell viability of prepared ADCs may be assessed using a CellTiter-Glo® Luminescent Cell Viability Assay, as described in the examples provided herein. In this assay, in certain aspects, the single reagent (CellTiter-Glo® Reagent) is added directly to cells cultured in serum-supplemented medium. The addition of reagent results in cell lysis and generation of a luminescent signal proportional to the amount of ATP present. The amount of ATP is directly proportional to the number of cells present in culture. The protein-binding dye sulforhodamine B (SRB) can also be used to measure cytotoxicity (Skehan et al. (1990) J Natl Cancer Inst. 82:1107-12).

In vivo assays that evaluate the promotion of tumor cell death and tumor schrinkage by mechanisms such as apoptosis may also be used. In one aspect, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

An exemplary aspect is a method of reducing or inhibiting growth of a tumor, comprising administering a therapeutically effective amount of a cytokine engager disclosed herein. In some aspects, the treatment is sufficient to reduce or inhibit the growth of the subject's tumor, reduce the number or size of metastatic lesions, reduce tumor load, reduce primary tumor load, reduce invasiveness, prolong survival time, and/or maintain or improve the quality of life.

In various aspects, treatment involves single bolus or repeated administration of the cytokine engager via an acceptable route of administration. In some aspects, the efficacy of a cytokine engager may be evaluated by contacting a tumor sample and a population of T cells (e.g., CD8⁺ T cells) from a subject with the cytokine engager and evaluating tumor growth rate or volume. In some aspects, when a cytokine engager has been determined to be effective, it may be administered to the subject.

### EXAMPLES

### Example 1: Design and Rationale for CD8-Binding Cytokine Engagers

CD8⁺ T cell activation and differentiation is in large part controlled by soluble immunomodulatory proteins such as cytokines. Cytokines typically have pleiotropic effects, causing multiple downstream cellular events such as activation, proliferation, survival, apoptosis, and secretion of other immunomodulatory proteins. In addition, because their receptors are expressed on multiple immune cell subsets, cytokines act not only on CD8+ T cells but also on other immune and non-immune cells that express their receptors (Cox M A et al. Trends Immunol. 2011; 32(4): 180-186). IL-2 is a potent cytokine that stimulates T and NK cell proliferation through either a heterotrimeric IL-2 receptor (IL-2R) composed of CD25, CD122 and CD132, or a heterodimeric IL-2 receptor composed of only CD122 and CD132 (Overwijk W W et al. Annu. Rev. Med. 2021; 72:281-311). Both forms of the IL-2R are potent mediators of T cell survival, proliferation, and overall activation status. IL-2 is generally produced by T cells and NK cells upon activation and mediates signaling in cis and trans in the local microenvironment. IL-2R signaling can induce differentiation of naïve T cells into effector and memory T cells and can also stimulate suppressive regulatory T cells.

Its ability to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma. However, there exist concerns in relation to the side effects produced by recombinant human IL-2 treatment. Patients receiving high-dose IL-2 treatment frequently experience severe cardiovascular, pulmonary, renal, hepatic, gastrointestinal, neurological, cutaneous, haematological and systemic adverse events, which require intensive monitoring and in-patient management. The majority of these side effects can be explained by the development of so-called vascular (or capillary) leak syndrome (VLS), a pathological increase in vascular permeability leading to fluid extravasation in multiple organs (causing e.g., pulmonary and cutaneous edema and liver cell damage) and intravascular fluid depletion (causing a drop in blood pressure and compensatory increase in heart rate).

Targeting CD8⁺ T cells represents an opportunity to widen the therapeutic window and uncouple toxicity from efficacy of IL-2 immunotherapy. CD8 guiding through the use of the CD8-binding cytokine engagers described herein provides preferential activation of CD8⁺ effector T cells and memory T cells, while avoiding significant activation of potentially suppressive (T_{reg}) or toxic (CD4⁺ or NK) cell populations.

The CD8-binding cytokine engagers described herein have been engineered with a number of features to improve the therapeutic index of targeted IL-2 immunotherapy. The CD8-binding cytokine engagers utilize cis(CD8)-guiding (i.e., anti-CD8 monoclonal antibody guiding) to maximize the potency of CD8⁺ T cell activation while maintaining the pMHC:TCR interaction upon CD binding. The CD8-binding cytokine engagers further utilize a human IgG1™ domain that confers extended pharmacokinetic parameters with low effector function. Additionally, bivalent IL-2 dosing and an optimized linker length (2xG4S) further modulate cytokine engager activity, in combination with IL-2 mutein selection (e.g., E61K) to further improve the therapeutic safety profile. As described herein, IL-2 mutein selection, such as the CD25 null mutein E61K, may limit undesirable T_{reg} cell activation following administration of the CD8-binding cytokine engagers. In parallel, the IL-2 mutein in the CD8-binding cytokine engager should minimize activation of IL2R-expressing cells (e.g., CD4⁺ T cells, NK cells and Eosinophils) to limit peripheral toxicities.

### Example 2: In Vitro Characterization of CD8-Binding Cytokine Engagers

Based on the engineering strategies for a CD8-binding cytokine engager with an optimal therapeutic index described in Example 1, several candidate cytokine engagers were generated and characterized. For CD8 targeting, the anti-CD8 humanized monoclonal antibody (mAb) clones TRX2 (Oxford Therapeutic Antibody Centre, Oxford University, Oxford, United Kingdom) and hOKT8 (eBioscience, Hatfield, UK) were evaluated. Three CD8-binding cytokine engagers were generated: IL20212, hOKT8-IL2 (also referred to herein as IL20226), and hOKT8-IL2 comprising an alternate VL germline (also referred to herein as IL20251). Each cytokine engager candidate comprised the bivalent IL-2 mutant E61K.

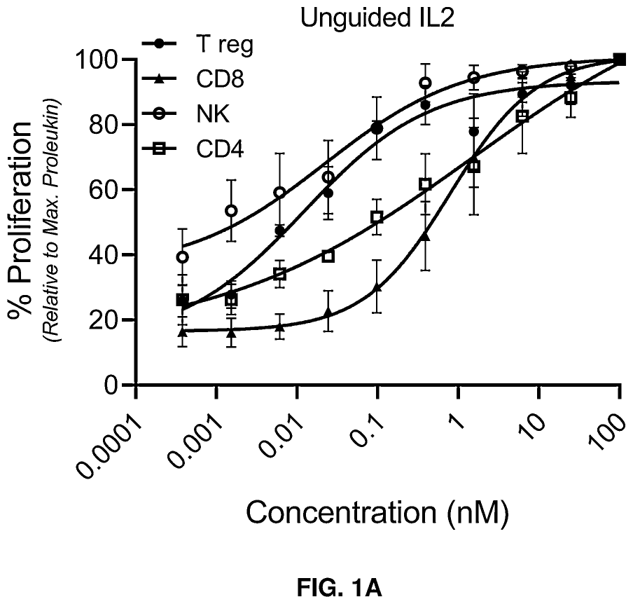

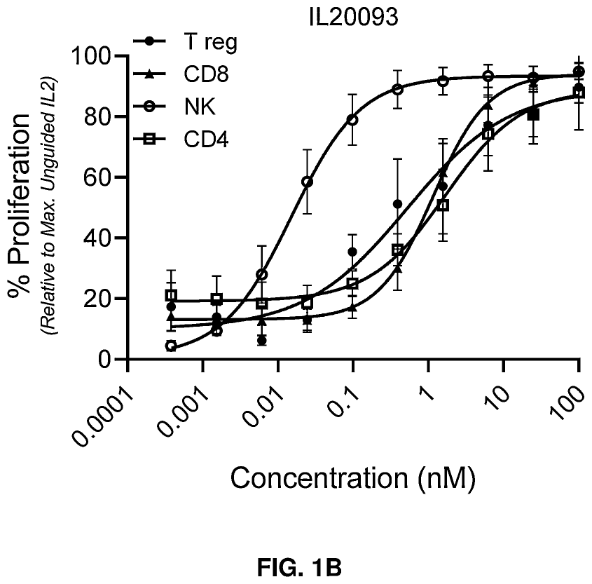

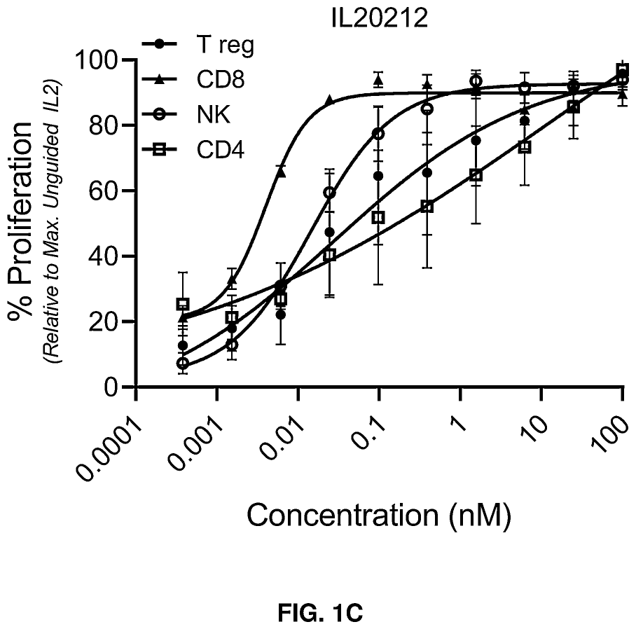

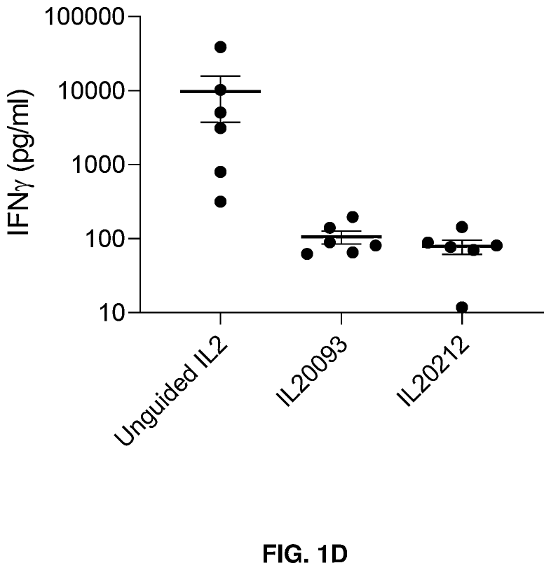

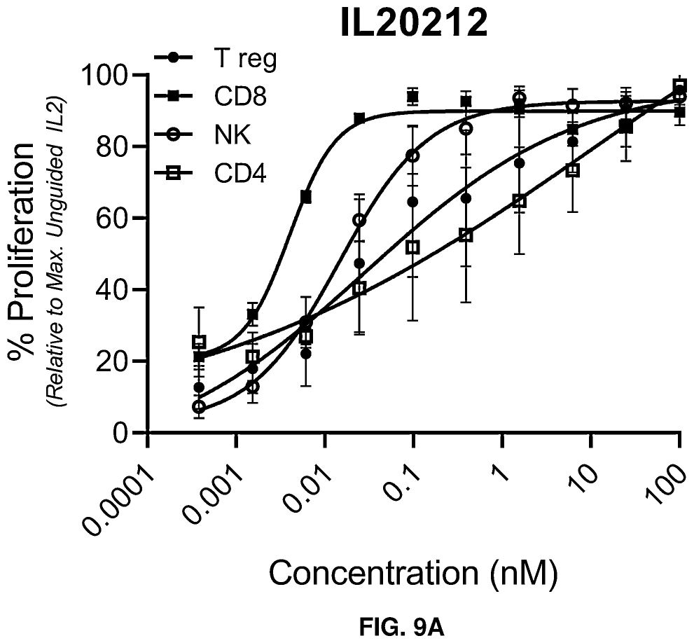

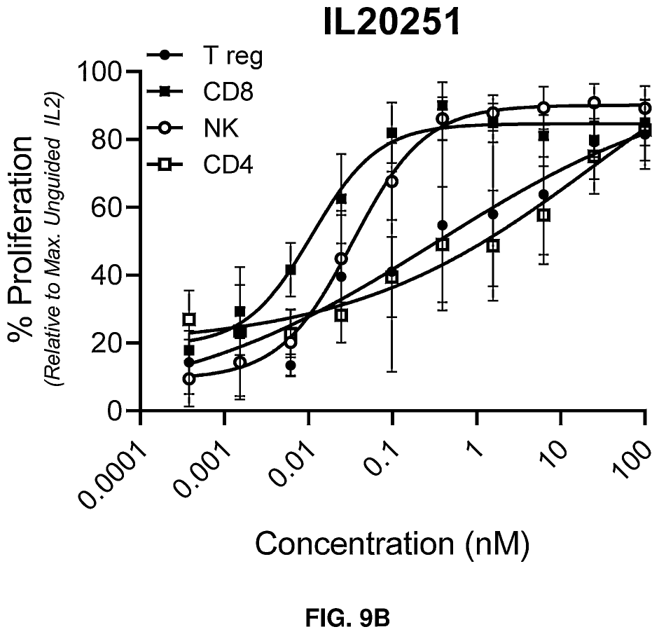

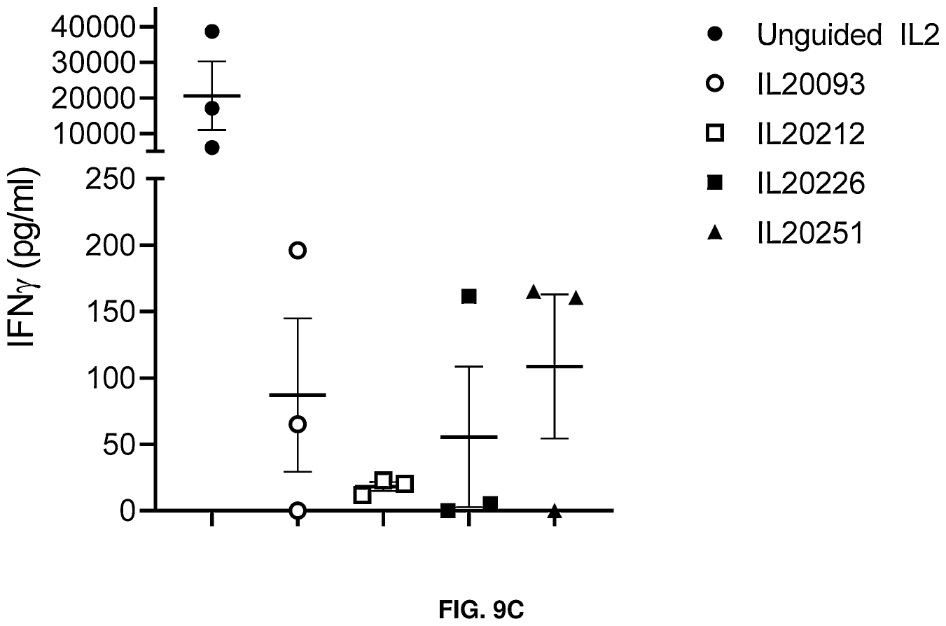

CD8⁺ T Cell Proliferation: The ability of the IL20212, IL20226, and IL20251 to induce proliferation of peripheral blood monocytes (PBMC) was examined and compared to unguided IL-2 and an isotype control cytokine engager (NIP228-IL2(E61K); also referred to herein as IL20093). PMBCs were treated with each molecule across a 10-fold dilution series from 0.0001 nM to 100 nM and cultured for 6 days. FIGS. 1A and 1B show the response of Tregs, CD8⁺ T cells, NK cells, and CD4⁺ T cells to unguided IL-2 and IL20093, respectively (shown as % proliferation relative to maximum unguided IL-2 response). FIGS. 1C and 9A-9C show the response of Tregs, CD8⁺ T cells, NK cells, and CD4⁺ T cells to IL20212, IL20226, and IL20251, respectively (shown as % proliferation relative to maximum unguided IL-2 response). IFNγ release (pg/mL) was also determined for each CD8-binding cytokine engager in comparison to unguided IL-2 and IL20093 (FIG. 9C), and between IL20212, unguided IL-2, and IL20093 (FIG. 1D), following treatment with each molecule (100 nM) and culture for 24 hours.

In response to each CD8-binding cytokine engager, CD8⁺ T cell proliferation was specifically augmented over Treg, NK cell, and CD4⁺ T cell proliferation. As shown in FIG. 1A, unguided IL-2 treatment led to nonspecific induction of NK cell, Treg, and CD4⁺ T cell proliferation at low concentrations, while only inducing CD8⁺ T cell proliferation at much higher doses (e.g., 1 to 10 nM). In contrast, the CD8-binding cytokine engagers shown in FIGS. 9A-9B led to robust CD8⁺ T cell proliferation at concentrations of 0.01 nM (IL20212) to 0.1 nM (IL20226 and IL20251), highlighting the advantage of targeted IL2(E61K) delivery to CD8⁺ T cells. As shown in FIGS. 1D and 9D, the CD8-binding cytokine engagers did not lead to a significant increase in IFNγ release, in contrast to unguided IL-2, which had a significant increase in IFNγ release.

Table 1 below shows the fold difference in dose required to induce proliferation of CD8⁺ T cells compared to Tregs, NK cells, or CD4⁺ T cells.

TABLE 1
Comparison of CD8-binding cytokine engager fold difference
in EC₅₀ dose to induce CD8⁺ T cell proliferation.

| Drug ID | CD8⁺/T_{reg} | CD8⁺/T_{reg} | CD8⁺/T_{reg} |
|---|---|---|---|
| IL20212 | −7 | −3.5 | −3057 |
| IL20226 | −3.5 | −4.3 | −248 |
| IL20251 | −32 | −3.3 | −2650 |
| IL20093 | 2.2 | 110 | −1.5 |
| Unguided IL-2 | 66.4 | 36.2 | −1.9 |

Given the superior EC₅₀ for inducing CD8⁺ T cell proliferation, the IL20212 CD8-binding cytokine engager was chosen for further in vitro characterization.

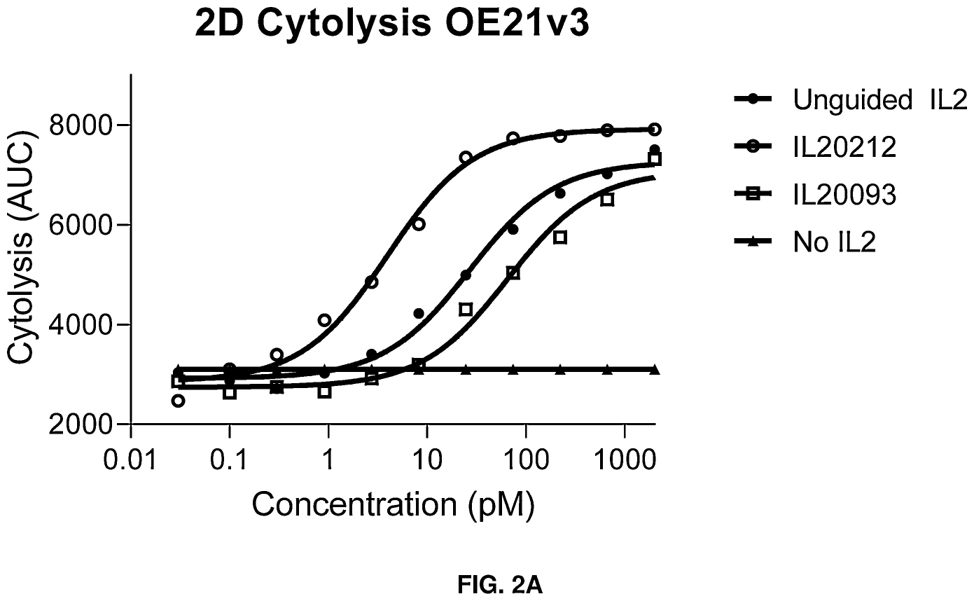

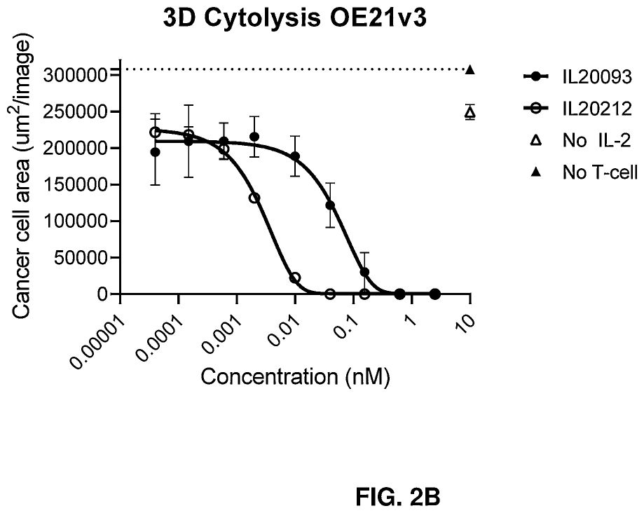

2D and 3D cytolysis: FIG. 2A show the effects of increasing concentrations of unguided IL-2, IL20093, IL20212, or a no IL2 control on 2D cytolysis and FIG. 2B show the effects of increasing concentrations of IL20093, IL20212, or a no IL2 control on 3D cytolysis (FIG. 2B) of an OE21 oesophageal cancer cell line engineered to express pp65 antigen (OE21v3). Briefly, pp65⁺ CD8 T cells were enriched within PBMC by expanding PBMC for 10 days with pp65 peptide and recombinant IL2. pp65 enriched PBMC were cultured in the presence of each drug and OE21v3 cells for 4 days before determining either the amount of cytolysis in 2D culture (xCelligence assay) or in 3D culture (Incucyte imaging). As shown in FIG. 2A, IL20212 cytokine engager treatment led to robust, dose-dependent cytolysis in 2D culture, and similarly, a significant decrease in 3D cancer cell area, as shown in FIG. 2B.

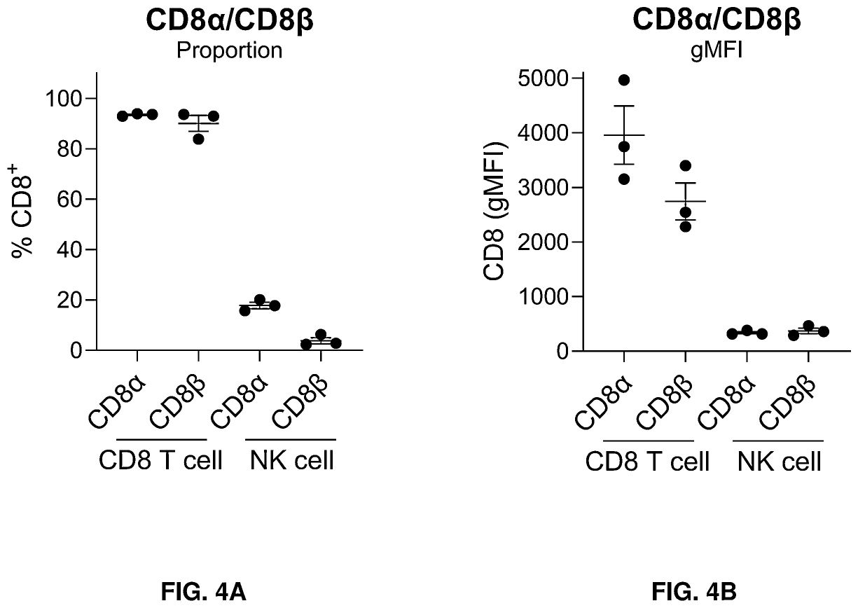

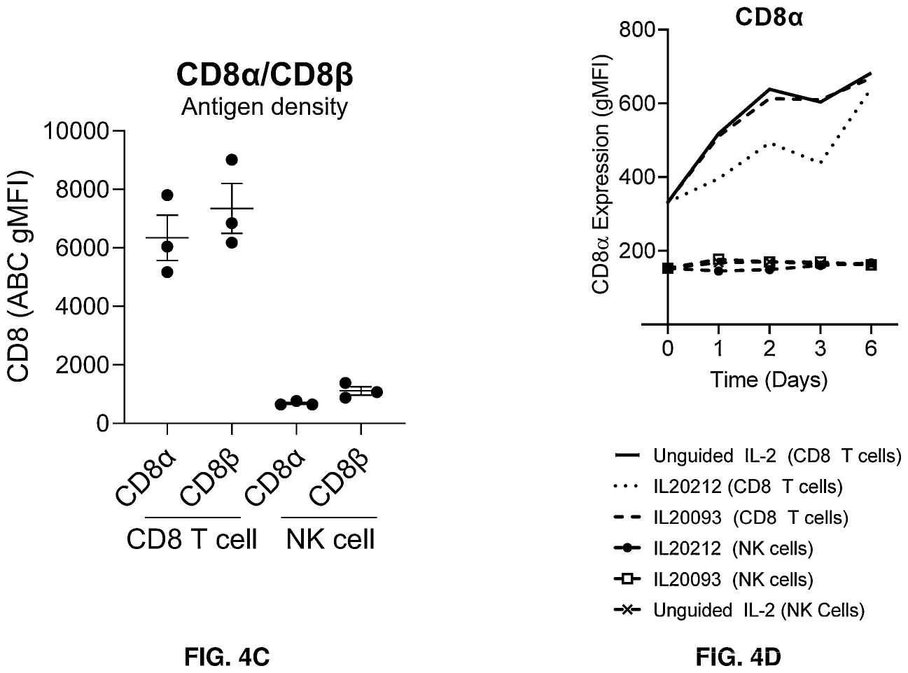

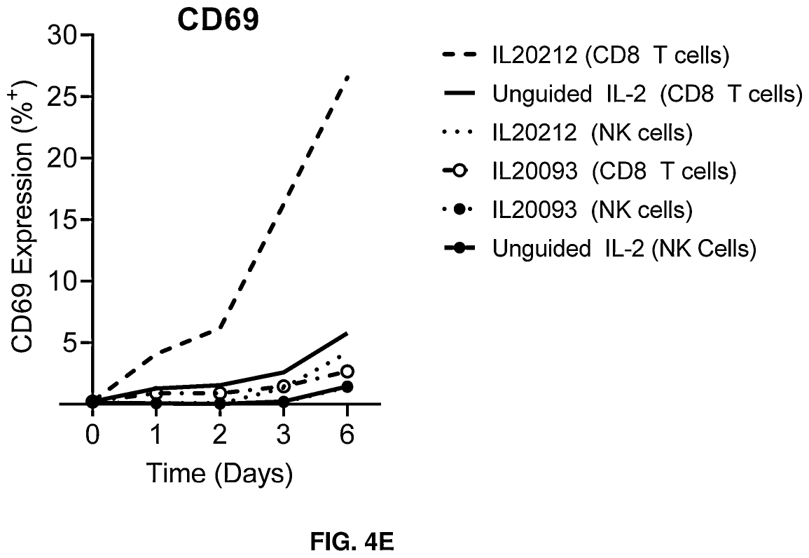

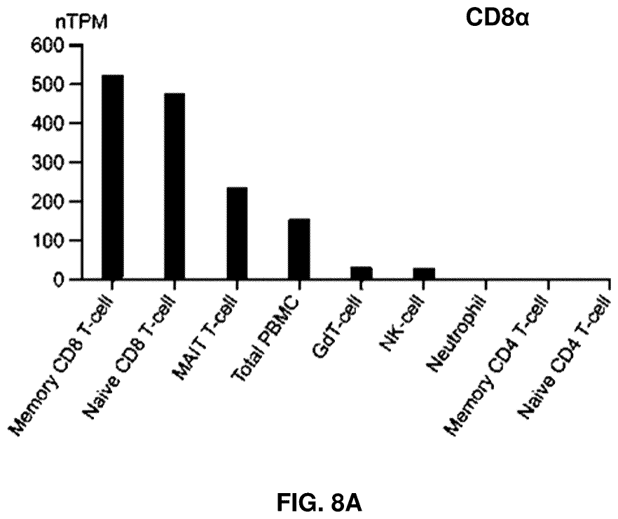

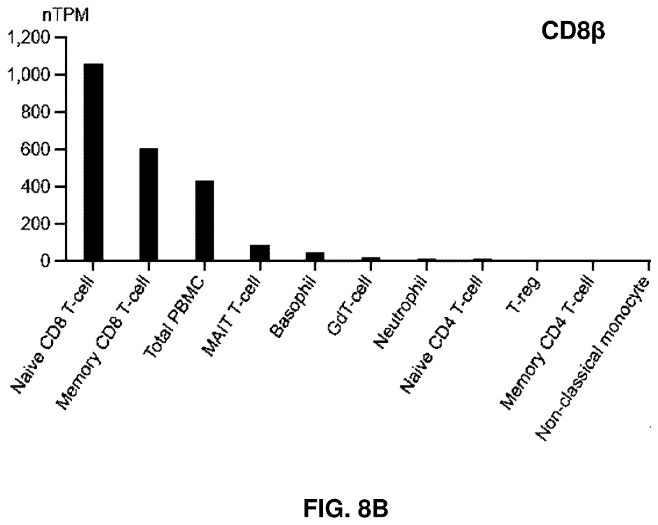

Assessing CD8α and CD8β expression in other cell types: As the CD8-binding cytokine engagers disclosed herein target the CD8α subunit, the levels of CD8α and CD8β expression in CD8⁺ T cells and NK cells were measured to assess the potential for NK cell activation. FIGS. 4A-4E show the expression of CD8α and CD8β in CD8⁺ T cells and NK cells at steady-state (shown as the proportion % positive) (FIG. 4A), gMFI (FIG. 4B), and surface antigen density (absolute number) (FIG. 4C). The expression of CD8α (FIG. 4D) and CD69 (FIG. 4E) in CD8⁺ T cells and NK cells in response to either unguided IL-2, NIP228-IL2, or CD8-IL2 treatment over time shown as gMFI. (N=3 in each test case). The results indicate that NK cells express limited CD8α at steady-state, with little evidence of increased CD8α expression or CD69 activation over-time. Expression of CD8α and CD8β across various cell types was analysed, as shown in FIGS. 8A-8B, respectively. CD8α expression levels are low in NK cells compared to CD8⁺ T cells (naïve and memory) as shown in FIGS. 8A-8B.

Comparison of IL20212 to clinical candidates: The IL20212 CD8-binding cytokine engager was compared to comparator molecules also implementing IL-2 targeting strategies for CD8⁺ T cell activation. IL20212 was compared to unguided IL-2, COMP2, and COMP1 for its ability to overcome Treg suppression. COMP2 is an IL-2R-beta-gamma targeted antibody-bivalent IL2 fusion protein wherein the cytokines are fused to LCDR3 of the antibody. COMP1 is a bispecific anti-PD-1 antibody fused to an IL-2 variant that does not bind IL-2Rα.

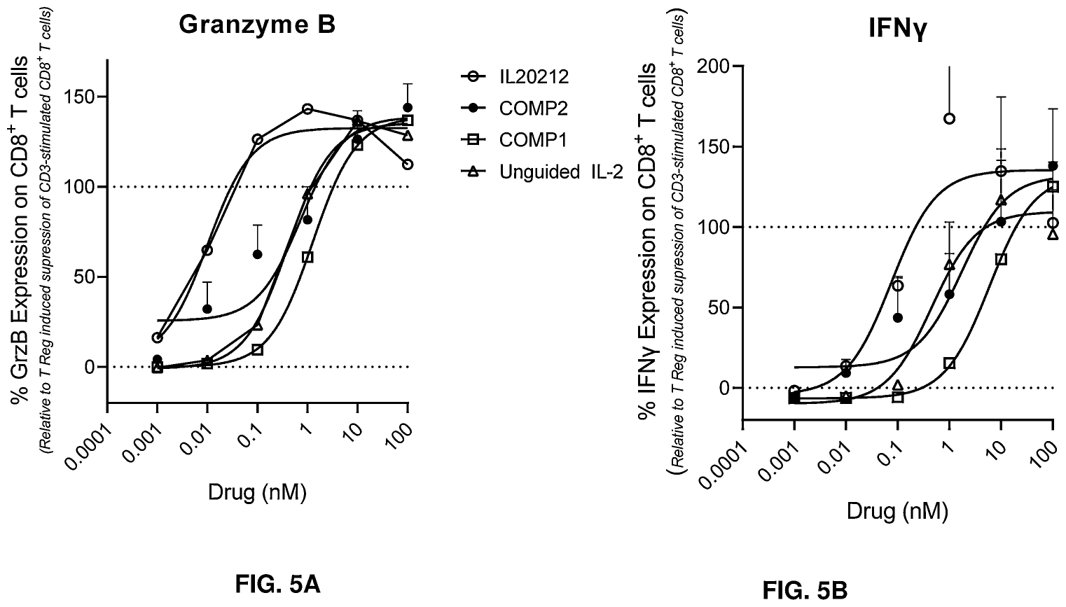

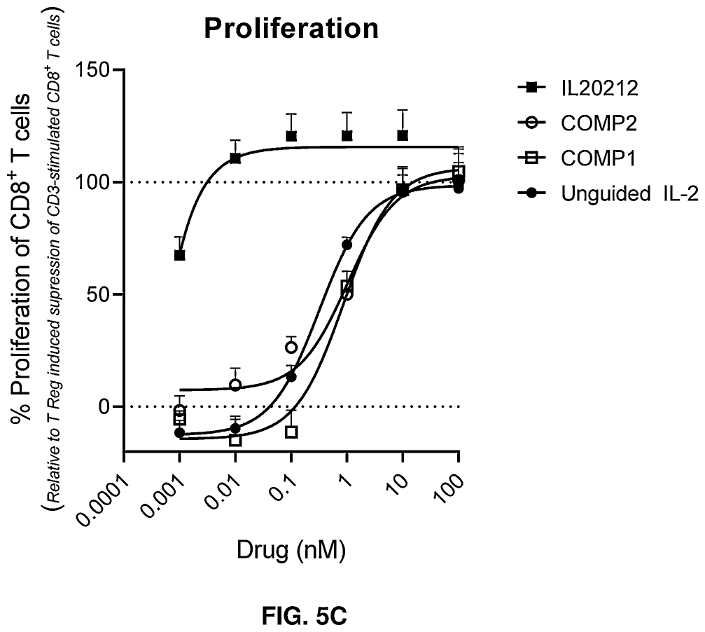

Briefly, Tregs were isolated from PBMC using CD25/CD127 selection, expanded for 6 days with anti-CD3 and recombinant IL2 and then cultured in the presented of T cell depleted PBMCs, effector T cell, 1 μg/mL anti-CD3 (OKT3), and the test drug (increasing 10-fold concentrations were tested for each compound from 0.001 nM to 100 nM) for 3 days. FIGS. 5A-5C show the effects of increasing concentrations of unguided IL-2, anti-CD8-IL2 (IL20212), anti-COMP1, and COMP2 treatment on CD8⁺ T cell granzyme B (GrzB) expression (FIG. 5A; shown as % GrzB expression of CD8⁺ T cells relative to Treg-induced suppression of CD3-stimulated CD8⁺ T cells), CD8⁺ T cell IFNγ expression (FIG. 5B; shown as % IFNγ expression of CD8⁺ T cells relative to Treg-induced suppression of CD3-stimulated CD8⁺ T cells), and CD8⁺ T cell proliferation (FIG. 5C; shown as % proliferation of CD8⁺ T cells relative to Treg-induced suppression of CD3-stimulated CD8⁺ T cells). IL20212 showed a superior ability to overcome Treg suppression as evidenced by the increase in GrzB and IFNγ expression over the other test drugs. IL20212 also led to significantly higher CD8⁺ T cell proliferation at every dose tested compared to COMP2 and COMP1, each of which had a similar effect as unguided IL-2 on CD8⁺ T cell proliferation.

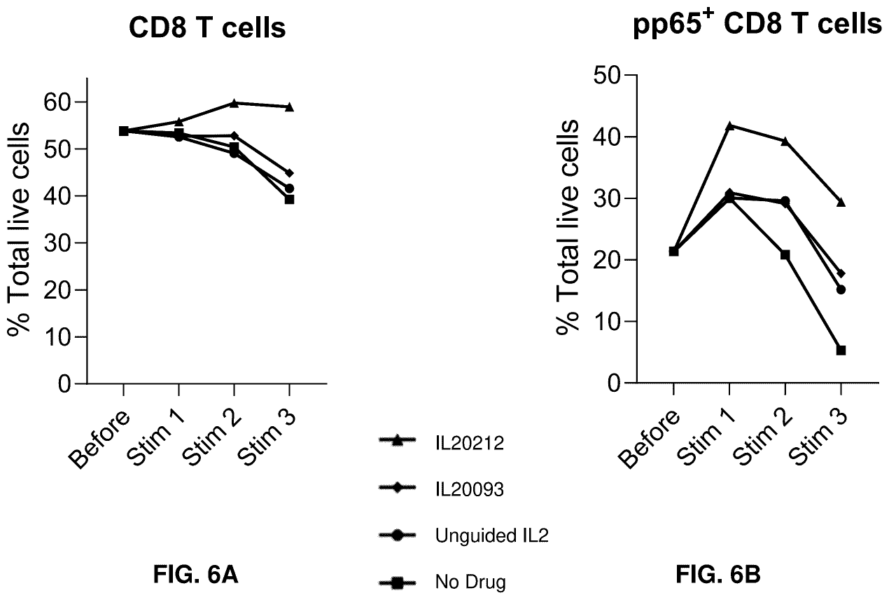

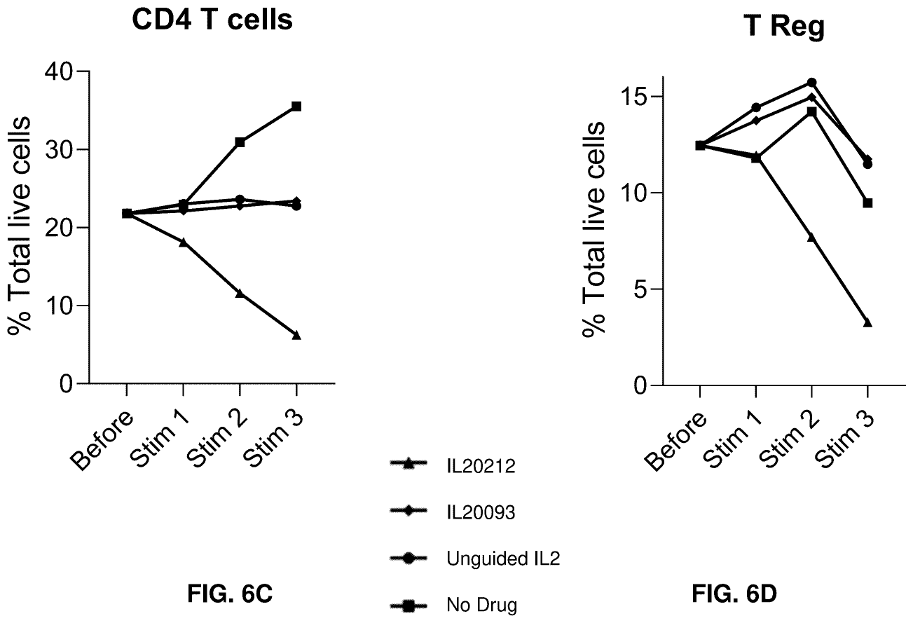

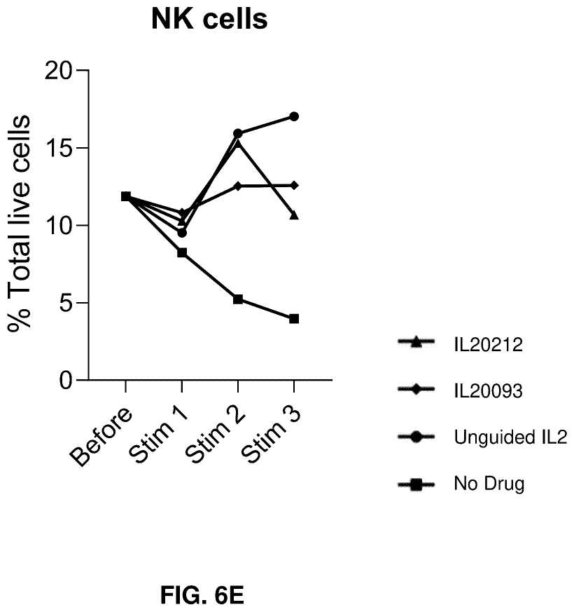

Effects on CD8⁺ T cell enrichment and Treg reduction: The ability of the IL20212 CD8-binding cytokine engager to enrich for CD8⁺ T cells while reducing Treg cells over time, following repeated administrations of the compound, was also assessed. Briefly, pp65⁺ CD8 T cells were enriched within PBMC by expanding PBMC for 10 days with pp65 peptide and recombinant IL2. pp65 CD8 T cell enriched PBMCs were cultured in the presence of OE21v3 and stimulated with 100 nM of either IL20212, IL20093, unguided IL-2, or in the absence of any drug for one (stim 1, duration 3 days), two (stim 2, duration 4 days), or three (stim 3, duration 3 days) stimulations. Following each stimulation, the frequency of different populations of cells was analyzed by FACS for the % of CD8⁺ T cells, pp65⁺ CD8⁺ T cells, CD4⁺ T cells, Tregs, and NK cells. FIGS. 6A-6E show the frequency of CD8⁺ T cells (FIG. 6A), pp65⁺ CD8⁺ T cells (FIG. 6B), CD4⁺ T cells (FIG. 6C), Tregs (FIG. 6D), and NK cells (FIG. 6E) as % total live cells in response to anti-CD8-IL2, NIP228-IL2, unguided IL-2, or no drug treatments. % total live cells are shown before treatment, and after one (stim 1), two (stim 2), or three (stim 3) doses of each drug. IL20212 was found to enrich for CD8⁺ T cells and reduce the proportion of Treg cells and CD4⁺ T cells over time.

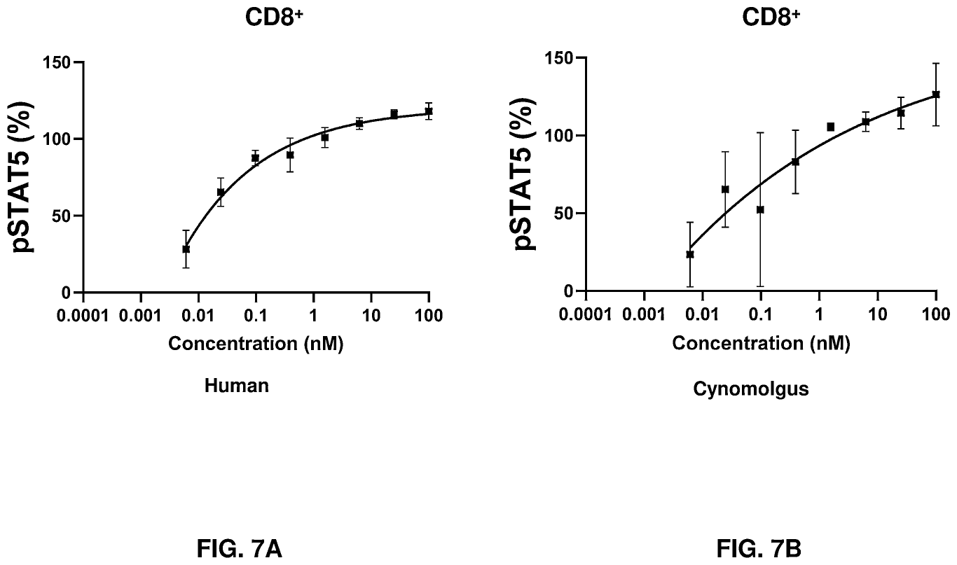

Cynomolgus cross-reactivity of IL20212: Cross-reactivity between human and cynomolgus CD8⁺ T cells was assessed by measuring pSTAT5 induction following IL20212 treatment. FIGS. 7A-7B show the % of pSTAT5 signaling on human CD8⁺ T cells (FIG. 7A) and cynomolgus CD8⁺ T cells (FIG. 7B) in response to increasing concentrations of anti-CD8-IL2 treatment. IL20212 shows cynomolgus cross-reactivity, with comparable dose-dependent pSTAT5 activation to human CD8⁺ T cells. Binding of IL20212 to human and cynomolgus CD8α/β antigens was also determined by surface plasmon resonance (SPR) binding analysis. As shown in Table 2, a lower K_{D} was observed for IL20212 binding to human CD8α/β than cynomolgus CD8α/β, although cross-reactivity is evident.

TABLE 2
IL20212 activation and binding of human and cynomolgus CD8

| | |
|---|---|
| Human | Cynomolgus |

| | | | | |
|---|---|---|---|---|
| Compound | CD8 | CD8α/β | CD8 | CD8α/β |
| | pSTAT5 | K_{D} | pSTAT5 | K_{D} |
| | (EC₅₀) | (nM) | (EC₅₀) | (nM) |
| IL20212 | 20 pM | 9 | 83 pM | 1.5 |

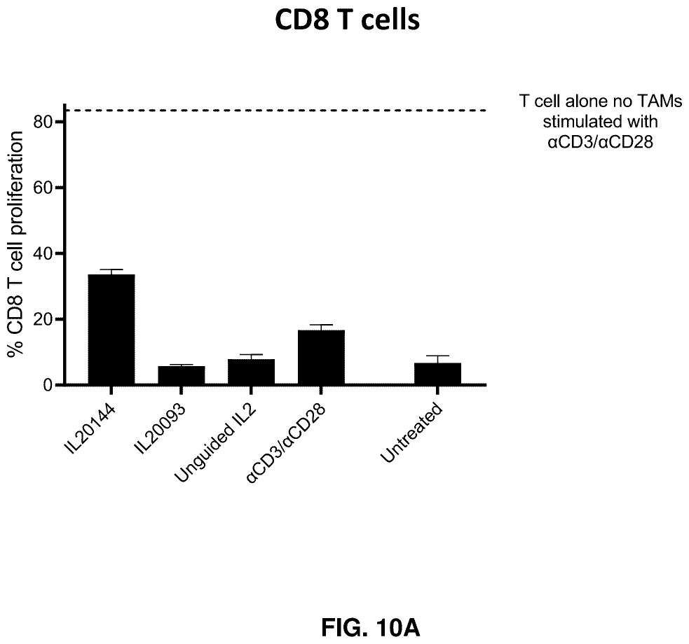

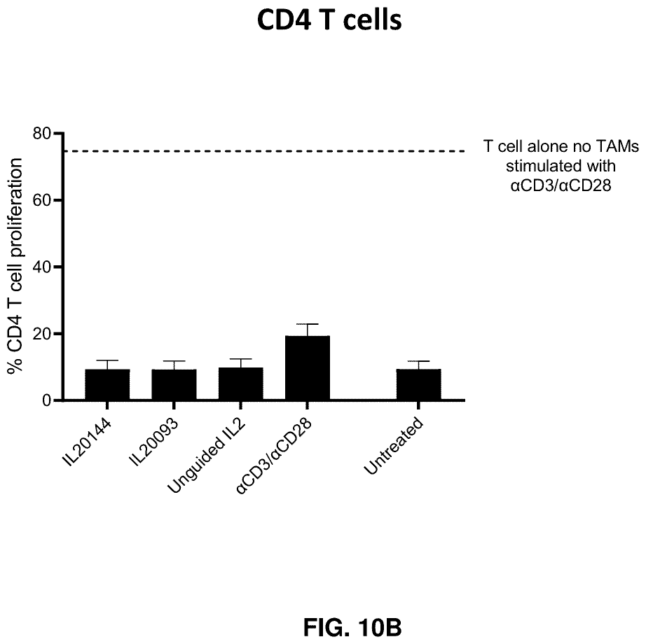

Rescue of T cell expansion: The ability of the IL20212 CD8-binding cytokine engager to rescue T cell expansion in the presence of suppressive tumor-associated macrophages (TAMs) was next determined. Briefly, monocytes were cultured in the presence of macrophage colony-stimulating factor (M-CSF) and MDA-MB-231 human breast cancer cell supernatant for 7 days to induce differentiation of the monocytes into TAMs. The TAMs were then cultured in the presence of T cells and drugs for 3 days. FIGS. 10A-10B show the effects of 1 nM IL20144, IL20093, unguided IL-2, and 1 ug/mL αCD3 plus 2 ug/mL αCD28 (polyclonal T cell stimulation) treatment on either CD8⁺ T cell proliferation (FIG. 10A) or CD4⁺ T cell proliferation (FIG. 10B) in the presence of suppressive tumor-associated macrophages (TAMs). IL20144 is an anti-CD8 cytokine engager comprising the parental antibody of IL20212, and also contains bivalent IL-2 with E61K. Maximal T cell proliferation is displayed as αCD3/αCD28 treatment in the absence of TAMs. IL20144 treatment led to an increase in CD8⁺ T cell proliferation in the presence of the TAMs, above all other treatments tested.

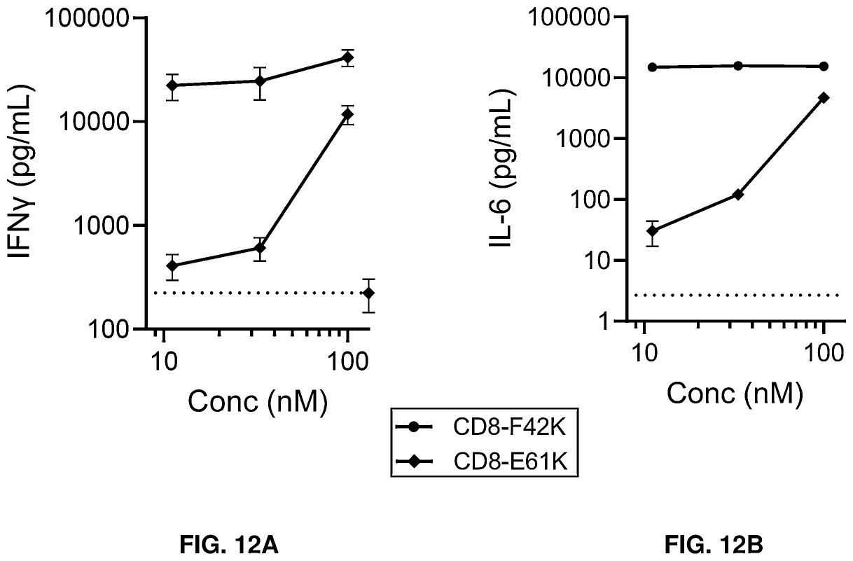

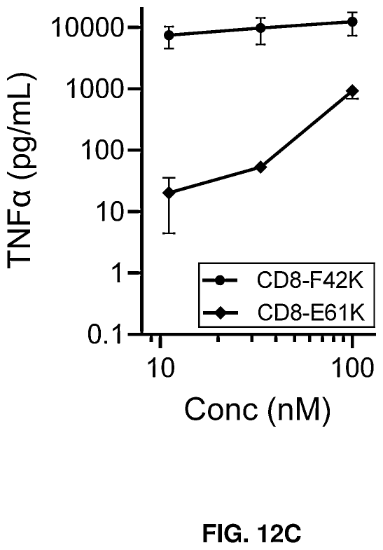

Comparison of monovalent and bivalent IL-2 cytokine engagers: IL2 muteins (E61K and F42K) were compared in the context of a monovalent CD8 diabody for pro-inflammatory cytokine secretion from PBMCs. Briefly, PBMCs were cultured in the presence of monovalent anti-CD8-E61K or anti-CD8-F42K diabody (no Fc tethering) for 24 hours, followed by ELISA for IFNγ, TL-6, and TNFα. FIGS. 12A-12C show the effects of monovalent anti-CD8 diabody with either F42K-IL2 mutein or E61K-IL2 mutein on IFNγ secretion (FIG. 12A), IL-6 secretion (FIG. 12B), and TNFα secretion (FIG. 12C) from PBMC at three different concentrations. In each assay, the monovalent CD8-IL2(E61K) diabody led to reduced pro-inflammatory cytokine secretion from PBMCs compared to the CD8-IL2(F42K) diabody.

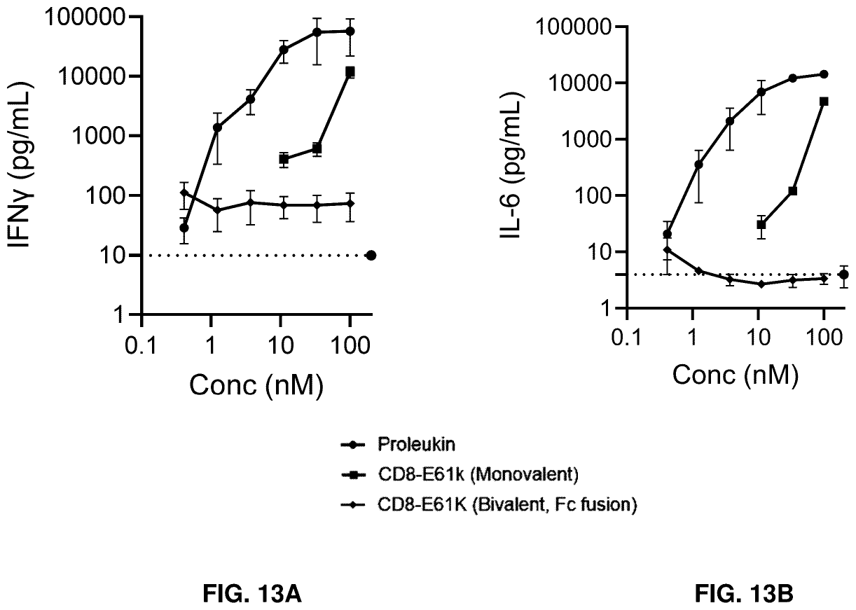

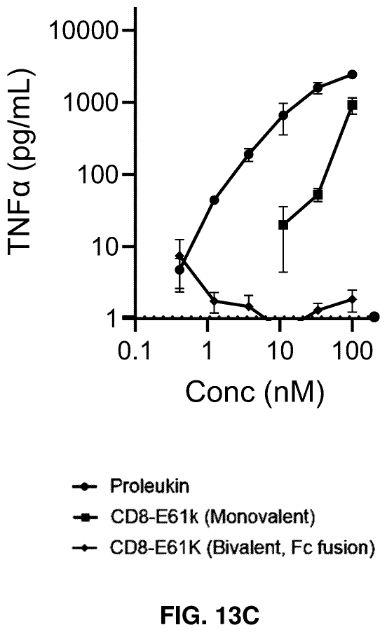

FIGS. 13A-13C compare the effects of unguided IL-2, monovalent anti-CD8-IL2(E61K), and bivalent anti-CD8-IL2(E61K) on IFNγ secretion (FIG. 13A), IL-6 secretion (FIG. 13B), and TNFα secretion (FIG. 13C) from PBMCs at increasing concentrations (0.1, 1, 10, and 100 nM) of each compound. Reduced IFNγ, IL-6, and TNFα release was observed in the context of the bivalent molecule compared to the monovalent molecule.

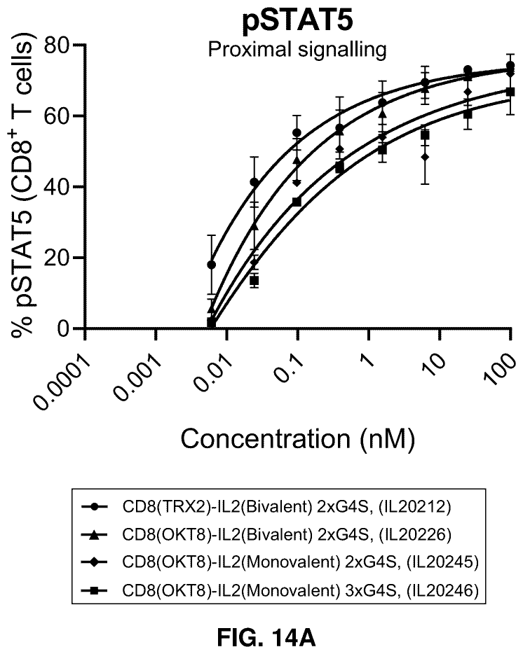

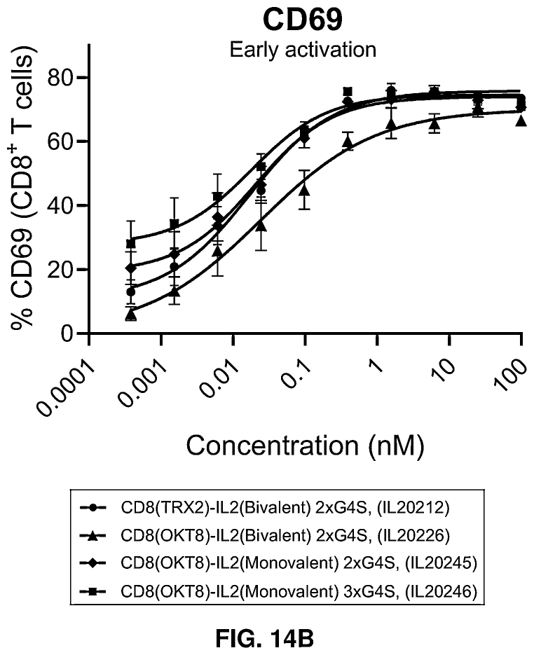

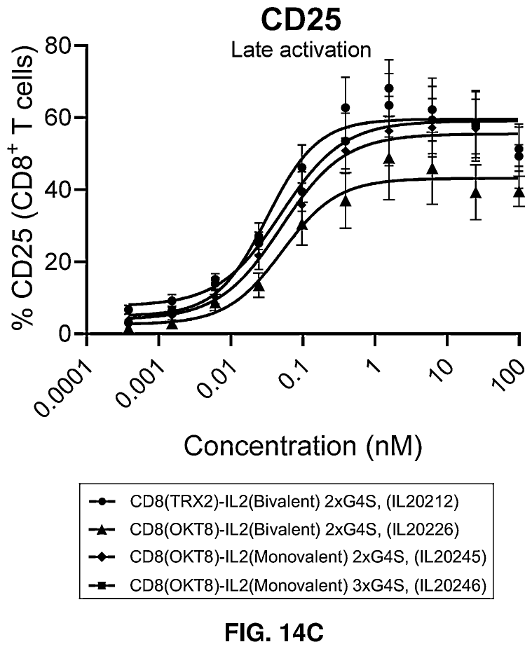

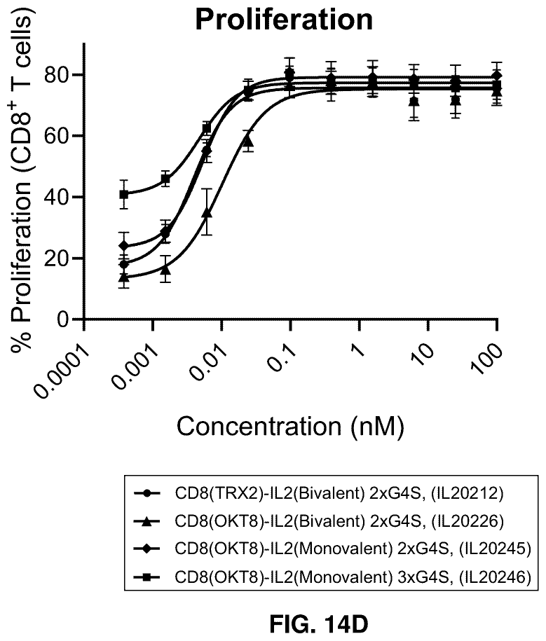

FIGS. 14A-14D show a comparison of bivalent anti-CD8-IL2 (IL20212), bivalent anti-CD8-IL2 (IL20226), monovalent anti-CD8-IL2 (IL20245), monovalent anti-CD8-IL2 (IL20246) on % pSTAT5 (FIG. 14A), % CD69 (FIG. 14B), % CD25 (FIG. 14C) expression, and proliferation of CD8⁺ T cells (FIG. 14D). 2xG4S indicates two repeats of a Gly-Gly-Gly-Gly-Ser linker between the IL2 and anti-CD8 antibody, and 3xG4S indicates three repeats of the same.

### Example 3: In Vivo CD8-Binding Cytokine Engager Evaluation

The IL20212 CD8-binding cytokine engager was tested in several in vivo mouse studies to determine efficacy in tumor killing, establish a safety profile. Studies were also performed to evaluate the effects of linker length on efficacy.

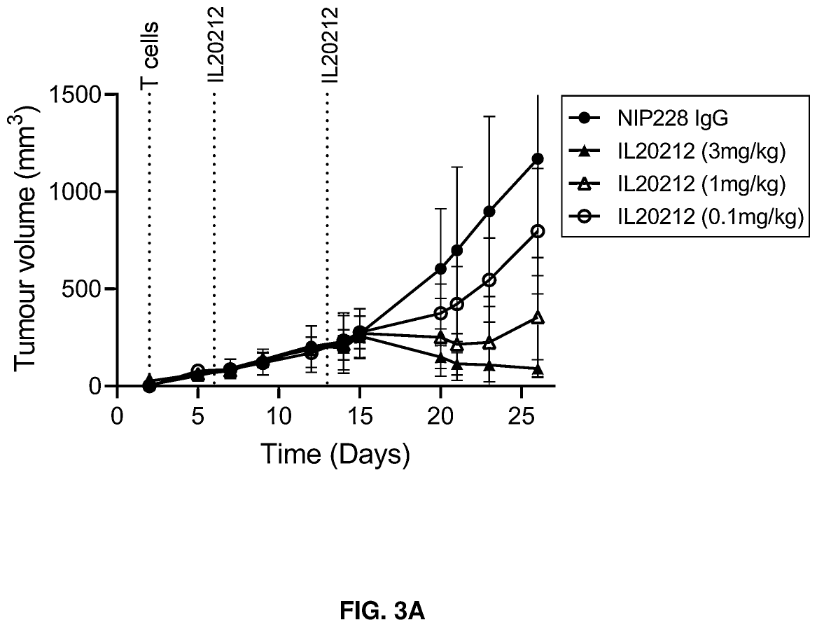

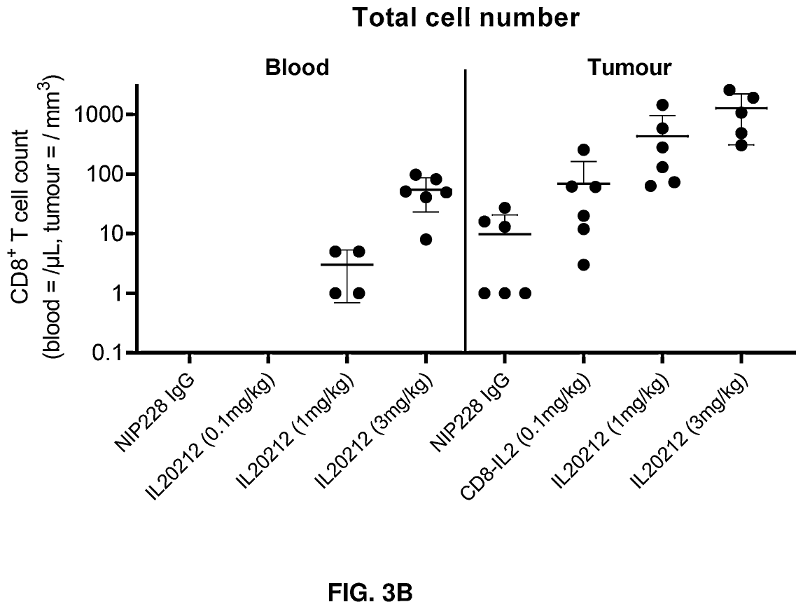

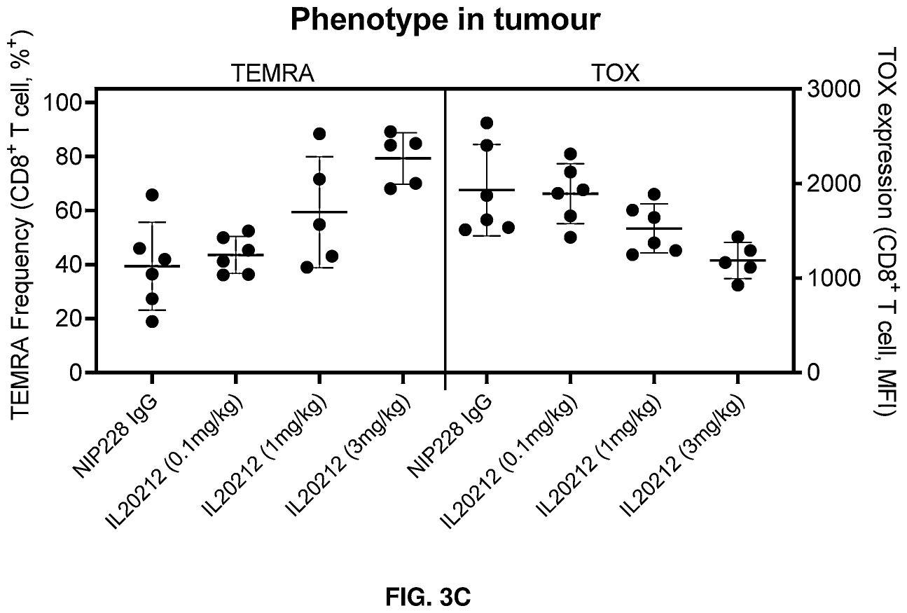

CD8 persistence and tumor growth inhibition: NSG mice were engrafted with 1×10⁶ pp65 enriched CD8+ T cells (multi-donor) in the presence of subcutaneously (s.c.)—injected pp65-expressing OE21v3 tumor cells. FIGS. 3A-3C show the effects of NIP228 IgG control or anti-CD8-IL2 treatment (at either 0.1, 1, or 3 mg/kg) on pp65-expressing OE21v3 tumor growth. Tumor volume across the duration of the study is shown in FIG. 3A. The results show that IL20212 was well tolerated for 2× doses at 3 mg/kg QW and led to significant and sustained reduction in tumor growth.

The total cell number of CD8⁺ T cells in the blood (shown as cells per μL blood) and tumor (shown as cells per mm³) for each treatment was also measured. As shown in FIG. 3B, on day 27, an increase in CD8⁺ T cell number was seen in the blood with 1 mg/kg and 3 mg/kg of IL20212. In the tumor, IL20212 at a dose of 0.1 mg/kg up to 3 mg/kg saw an increase in CD8⁺ T cell number compared to isotype control NIP228 IgG.

The tumor-infiltrating CD8⁺ T cell phenotype for each treatment group with respect to TEMRA (effector memory RA cells) frequency (as % of CD8⁺ T cells) and TOX (thymocyte selection-associated HMG BOX) expression in CD8⁺ T cells (shown as mean fluorescent intensity) was assessed. As shown in FIG. 3C, IL20212 treatment at 0.1, 1, and 3 mg/kg led to an increase in TEMRA and a decrease in TOX.

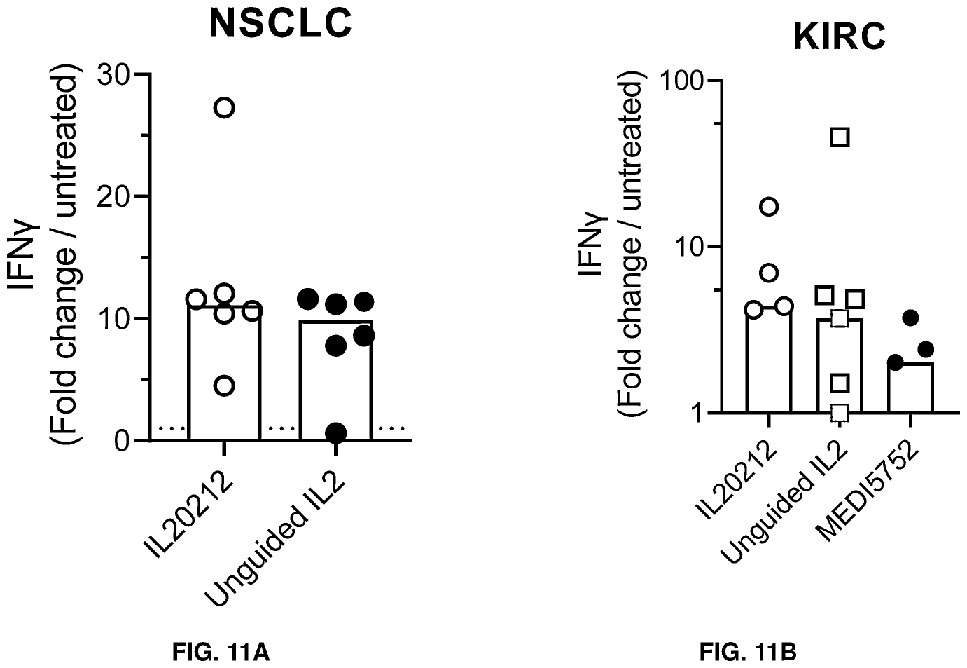

Activation of tumor-infiltrating lymphocytes (TILs): The ability of IL20212 to activate TILs to increase IFNγ secretion from primary tumor slices was examined. Fresh tumors were obtained from human patients with non-small cell lung cancer (NSCLC) and renal cell carcinoma (KIRC). Less than 24 hours after the tumors were resected from the patients, the tumor was embedded in agarose, sliced into tissue sections, and cultured for 3 days in the presence of the test drug. FIGS. 11A-11B show the effects of anti-CD8-IL2 and unguided IL-2 treatment on IFNγ secretion in non-small cell lung cancer (NSCLC) (FIG. 11A) and anti-CD8-IL2, unguided IL-2, and MEDI5752 treatment on IFNγ secretion in kidney renal cell carcinoma (KIRC) (FIG. 11B). The results show that IL20212 increased IFNγ secretion in NSCLC and KIRC tumor slices compared to untreated tumors. The increase in IFNγ was comparable to unguided IL-2 (NSCLC and KIRC) and MEDI5752 treatment (KIRC).

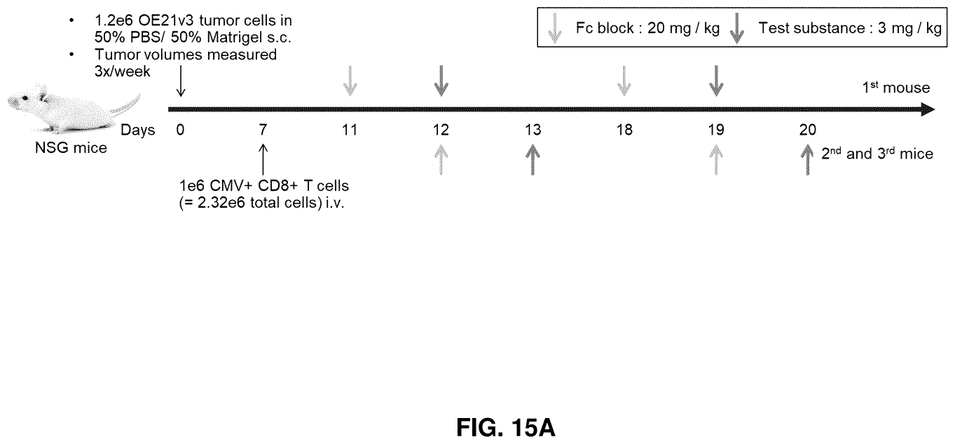

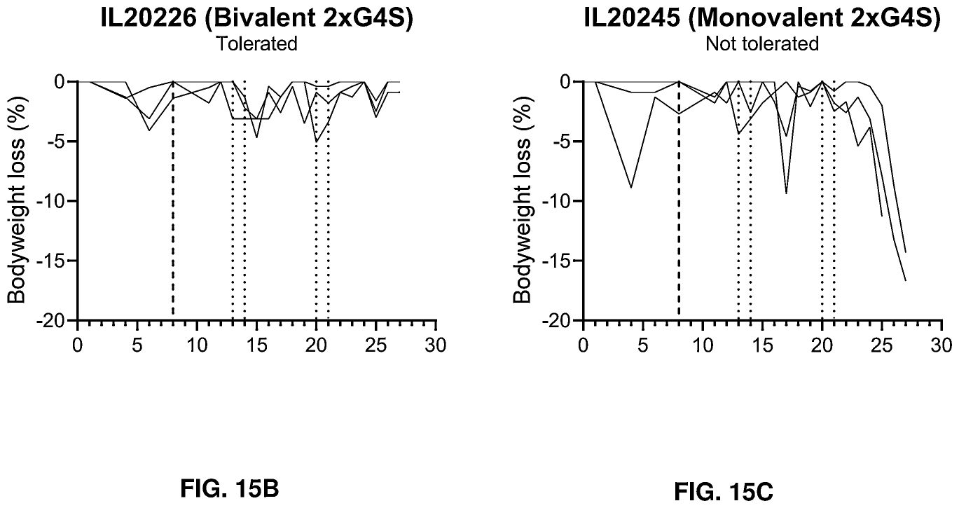

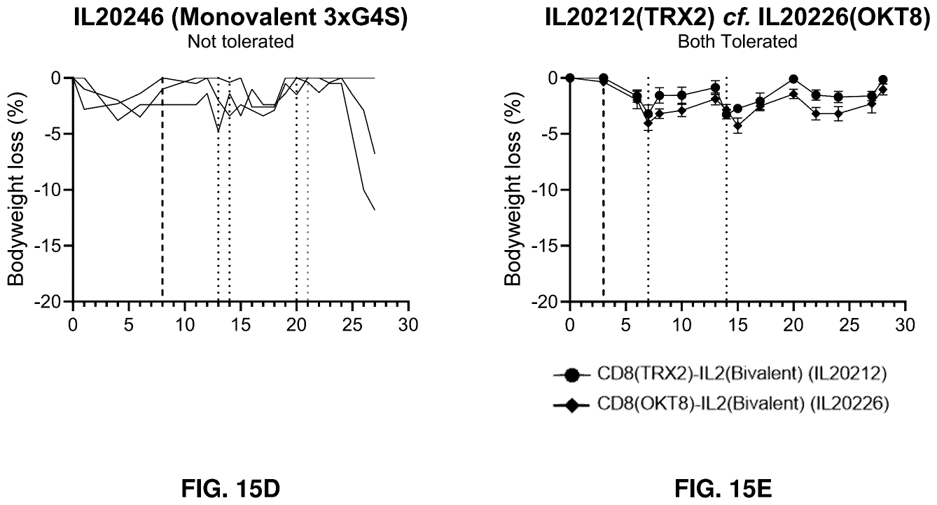

Comparison of bivalent and monovalent CD8-binding cytokine engagers: The safety profile of bivalent IL2 and monovalent IL2 drugs at 3 mg/kg was compared in NSG mice humanized pp65⁺ CD8⁺ T cell enriched PBMCs. FIG. 15A shows a schematic outlining the NSG mouse study, wherein on day 0 mice were injected subcutaneously (s.c.) with 1.2×10⁶ OE21v3 tumor cells in 50% phosphate-buffered saline (PBS)/50% Matrigel, and on day 7 injected intravenously (i.v.) with 1×10⁶ pp65+CD8+ T cell enriched PBMCs pre-expanded for 10 days in vitro (2.32×10⁶ total cells). An Fc block (20 mg/kg) was administered i.p. to a first mouse on days 11 and 18, and to a second and third mice on days 12 and 19. The test/drug substance (3 mg/kg) was administered i.p. to the first mouse on days 12 and 19, and to the second and third mice on days 13 and 20. The study endpoint was day 27. Bodyweight loss (calculated as a % of starting maximum bodyweight) was measured for mice treated with IL20226 (bivalent anti-CD8-IL2 with a 2xG4S linker) (FIG. 15B), IL20245 (monovalent anti-CD8-IL2 with a 2xG4S linker) (FIG. 15C), and IL20246 (monovalent anti-CD8-IL2 with a 3xG4S linker) (FIG. 15D). FIG. 15E shows the % bodyweight loss for mice treated with either bivalent anti-CD8-IL2 (IL20212) or bivalent anti-CD8-IL2 (IL20226). The results indicate that the bivalent CD8-binding cytokine engager with a 2xG4S peptide linker was well tolerated, with no significant change in body weight. In contrast, the monovalent IL20245 and IL20246 led to rapid weight loss following the second dose.

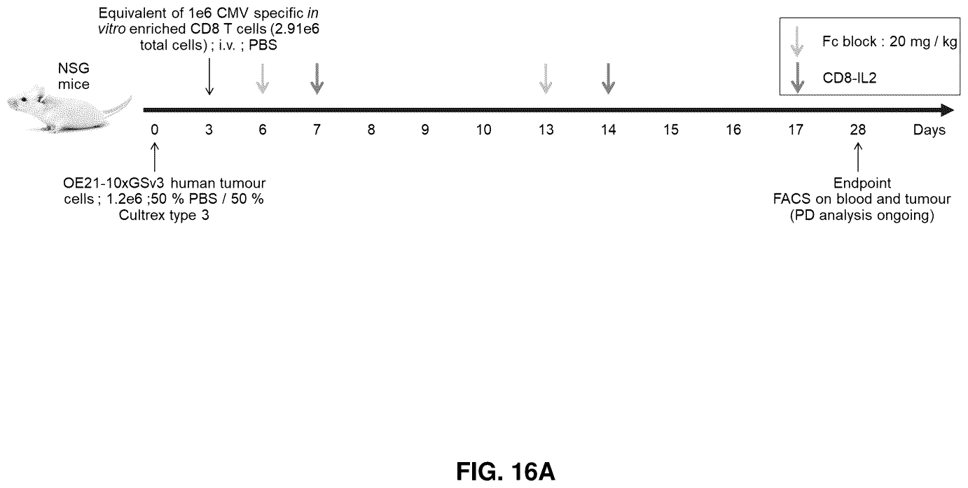

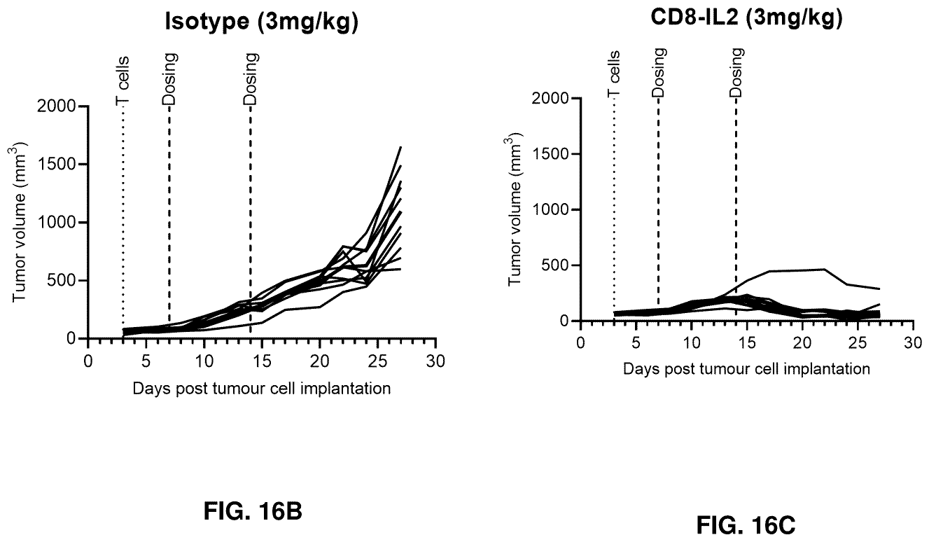

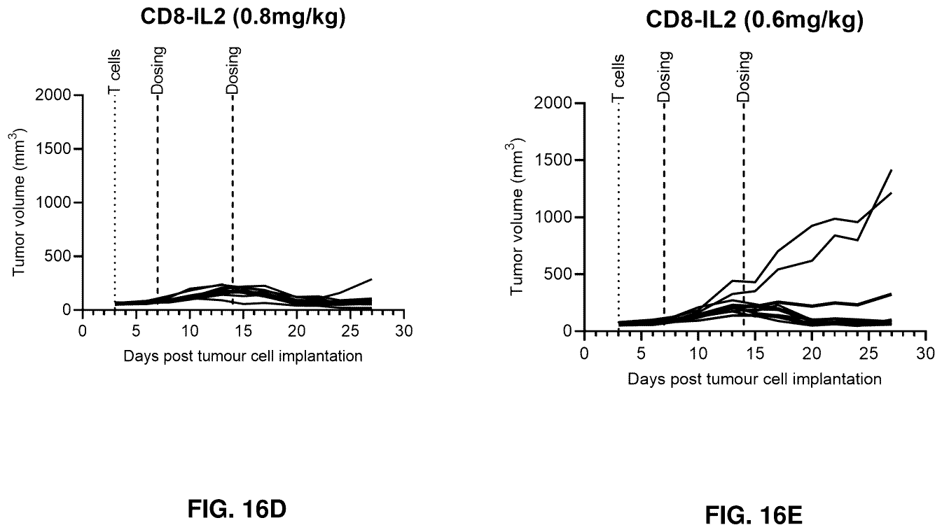

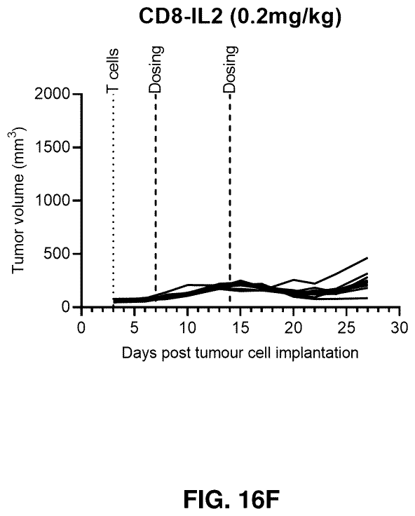

IL20212 dose titration and in vivo tumor growth: Several doses of IL20212 were tested in NSG mice with OE21 tumors. FIG. 16A shows a schematic outlining the NSG mouse study, wherein on day 0 mice were injected subcutaneously (s.c.) with 1.2×10⁶ OE21v3 tumor cells in 50% PBS/50% Cultrex type 3, and on day 3 injected intravenously (i.v.) pp65+CD8+ T cell enriched PBMCs pre-expanded for 10 days in vitro (2.91×10⁶ total cells). An Fc block (20 mg/kg) was administered i.p. on days 6 and 13, IL20212 was administered i.p. on days 7 and 14 at a dose of 3, 0.8, 0.6, and 0.2 mg/kg, or an isotype control was administered (3 mg/kg). The study endpoint was day 28. Tumor volume was measured beginning at day 3 of the study for each of the isotype control, and IL20212 treatments at 3, 0.8, 0.6, and 0.2 mg/kg dosages, as shown in FIGS. 16B-16F, respectively. The data indicate that OE21v3 tumors were well controlled with a dose of IL20212 of between 0.2 to 3 mg/kg.

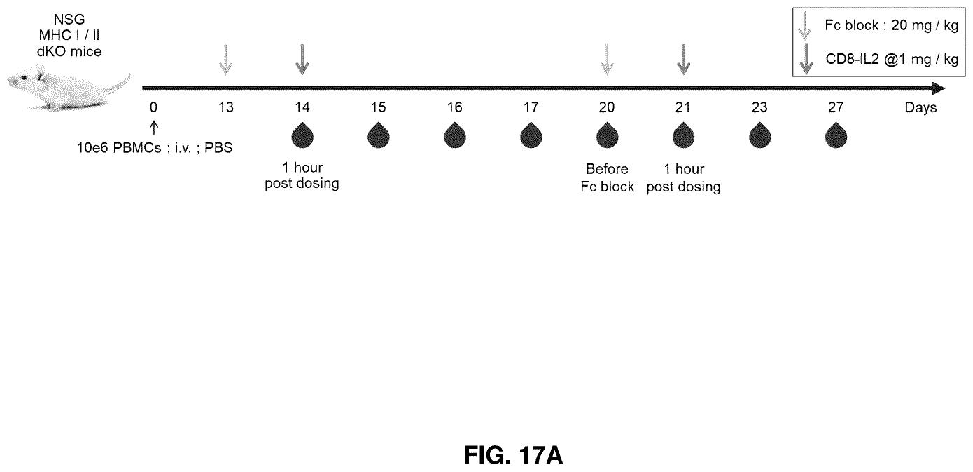

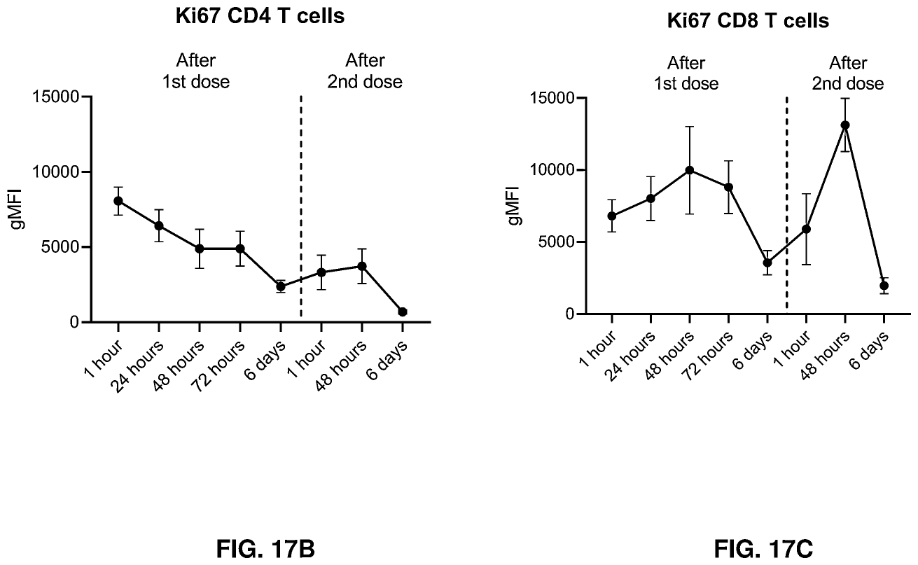

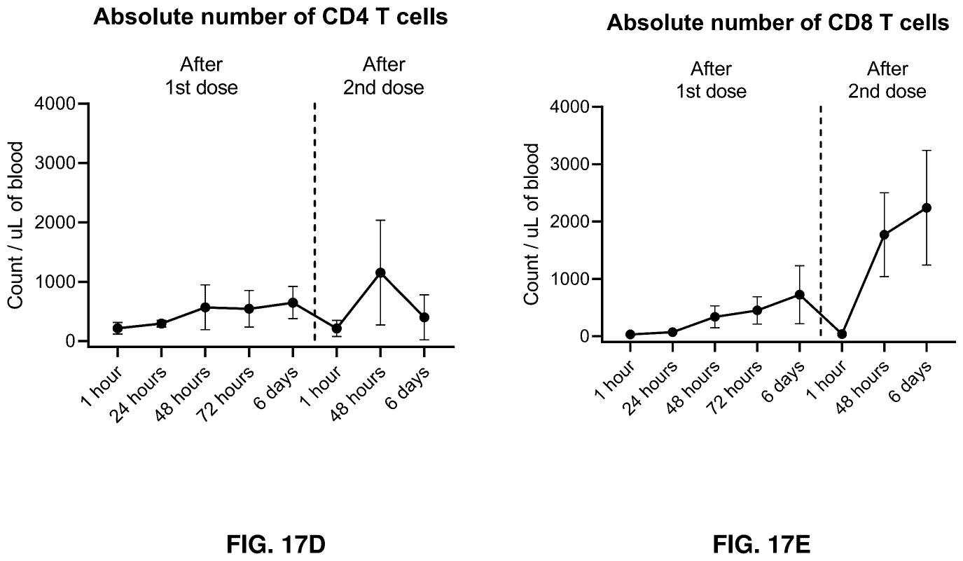

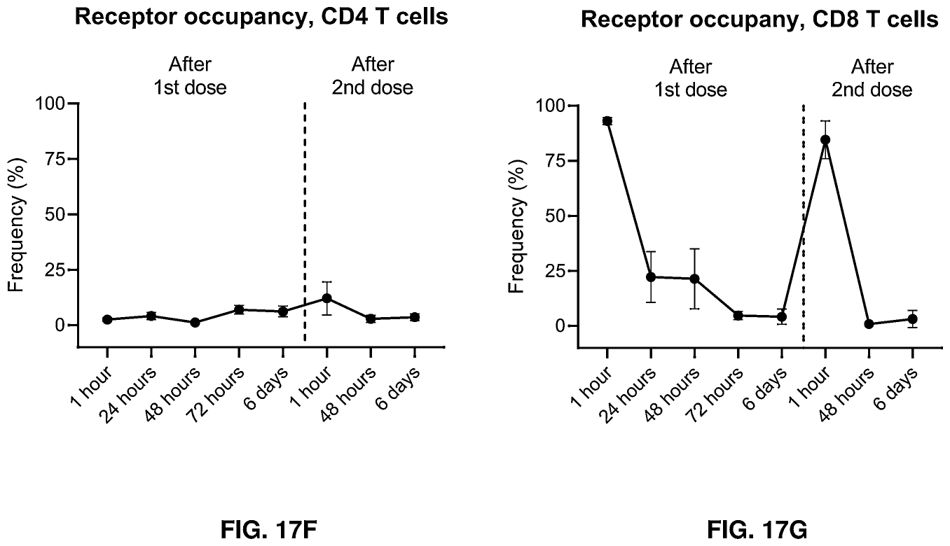

IL20212 expansion of CD8⁺ T cells in vivo: The ability of IL20212 to preferentially expand CD8⁺ T cells in vivo was assessed. FIG. 17A shows a schematic outlining the NSG MHCI^{-/-}/II^{-/-} mouse study, wherein on day 0 mice were injected intravenously (i.v.) with 1×10⁶ PBMCs in PBS, and on days 13 and 20 administered an Fc block (20 mg/kg). IL20212 was administered intraperitoneally (1 mg/kg) on days 14 and 21). Blood draws were performed each day from day 14 through day 27 of the study. Blood draws on days 14 and 21 were performed 1 hour post-dosing, and on day 20 before the Fc block. Ki67 expression was measured by gMFI after the first and second doses of IL20212 at the timepoints shown for both CD4⁺ T cells (FIG. 17B) and CD8⁺ T cells (FIG. 17C). Absolute number of CD4⁺ and CD8⁺ T cells was also measured in parallel, as shown in FIGS. 17D-17E, respectively. The results indicate a significant and preferential expansion of CD8⁺ T cells over CD4⁺ T cells and are further outlined in Table 3 below.

TABLE 3
Comparison of absolute number of CD4/CD8 T cells in blood

| | Baseline | Fold increase | Fold increase | Max Increase |
|---|---|---|---|---|
| | (count/μL | cycle 1 (1 h to 6 | cycle 2 (1 h to 6 | from baseline |
| | of blood) | days post dose 1) | days post dose 2) | (Time zero) |
| CD4 | 219 | 3 | 5 | 5 |
| CD8 | 115 | 24 | 61 | 74 |

Peripheral on-cell receptor occupancy of IL20212 was also measured (anti-IgG capture reagent) for both CD4⁺ T cells (FIG. 17F) and CD8⁺ T cells (FIG. 17G). The results indicate that IL20212 binding is guided to CD8⁺ T cells in vivo.

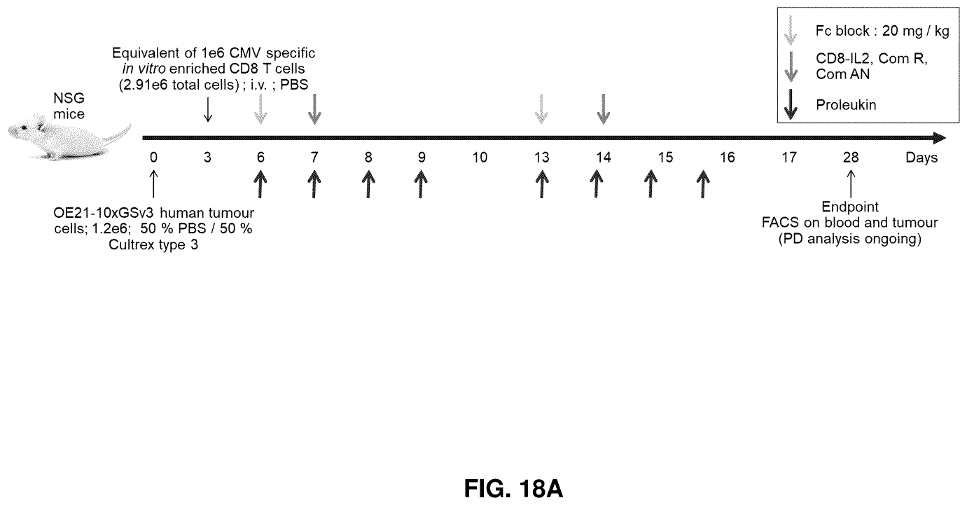

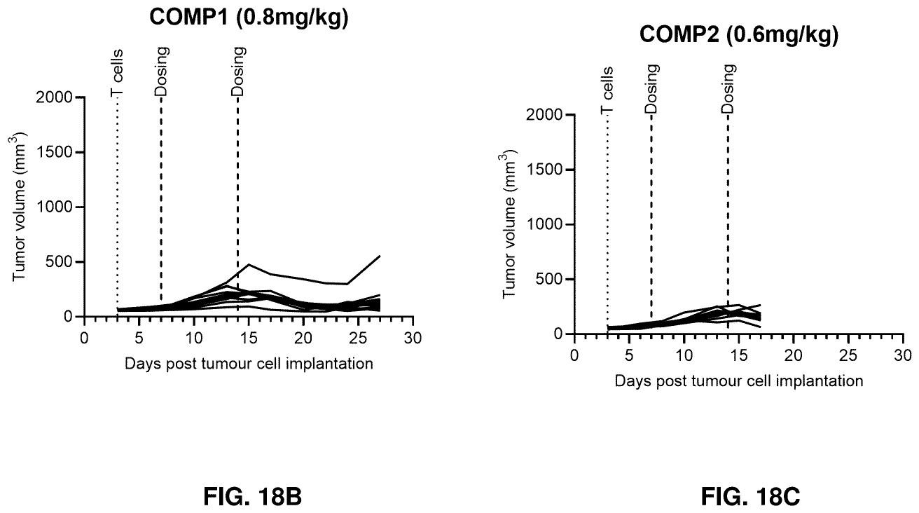

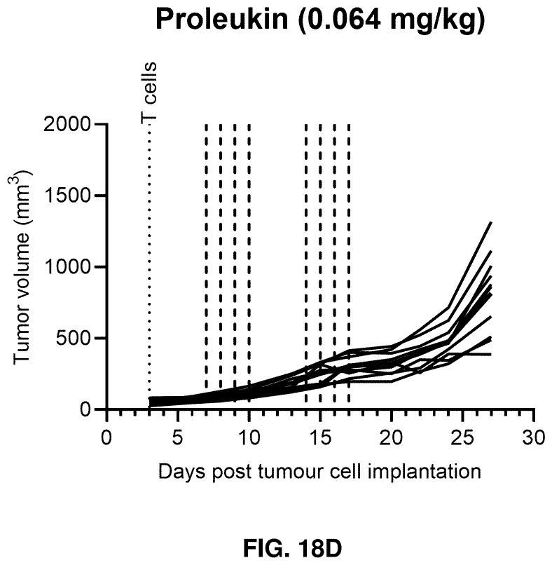

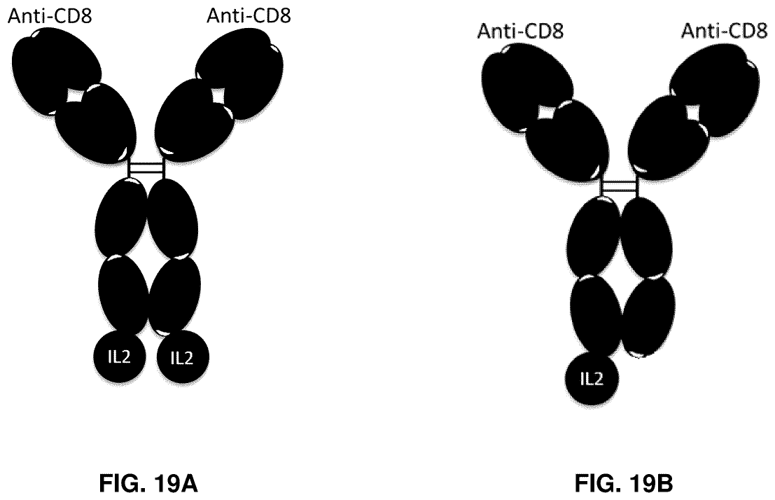

Comparison of IL20212 to comparators: IL20212 was compared to COMP1 and COMP2 for the ability to inhibit in vivo tumor growth. FIG. 18A shows a schematic outlining the NSG mouse study, wherein on day 0 mice were injected subcutaneously (s.c.) with 1.2×10⁶ OE21v3 tumor cells in 50% PBS/50% Cultrex type 3 (R&D Systems™), and on day 3 injected intravenously (i.v.) with pp65+CD8+ T cell enriched PBMCs pre-expanded for 10 days in vitro (2.91×10⁶ total cells). An Fc block (20 mg/kg) was administered i.p. on days 6 and 13, and IL20212 drug, COMP1, COMP2, unguided IL-2, or an isotype control were administered i.p. on days 7 and 14 (unguided IL-2 was administered on days 6-9 and 13-16). The study endpoint was day 28. Tumor volume was measured beginning at day 3 of the study for each of COMP1, COMP2, and unguided IL-2, as shown in FIGS. 18B-18D, respectively. Isotype control and IL20212 drug effects (0.8 and 0.6 mg/kg) on tumor volume are shown above in FIGS. 16B and 16D-16E.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All publications, patents, and patent applications disclosed herein are incorporated by reference to the same extent as if each individual publication, patent or patent application was specifically and individually indicated to be incorporated by reference.

1. A cytokine engager comprising an antibody that specifically binds cluster of differentiation 8 (CD8), wherein:
a. the antibody specifically binds to the CD8α chain of CD8; and
b. the antibody is linked to a cytokine.

2. (canceled)

3. The cytokine engager of claim 1, wherein the antibody comprises a heavy chain complementarity determining region (HCDR) 1, a HCDR2, and a HCDR3, comprising the amino acid sequence of SEQ ID NO: 6, SEQ ID NO: 7, and SEQ ID NO: 8, respectively; and a light chain complementarity determining region (LCDR) 1, a LCDR2, and a LCDR3, comprising the amino acid sequence of SEQ ID NO: 9, SEQ ID NO: 10, and SEQ ID NO: 11, respectively.

4. The cytokine engager of claim 1, wherein the antibody comprises a first heavy chain, a second heavy chain, and two light chains, and wherein:
a. either the first heavy chain or the second heavy chain of the antibody is fused at the C-terminus to the cytokine; or
b. the first heavy chain is fused at the C-terminus to the cytokine and the second heavy chain of the antibody is fused at the C-terminus to a second cytokine.

5. The cytokine engager of claim 1 wherein the antibody is fused to the cytokine and/or the second cytokine via a linker comprising SEQ ID NO: 13 or SEQ ID NO: 14.

6. (canceled)

7. The cytokine engager of claim 1, wherein the cytokine and/or the second cytokine is interleukin-2 (IL-2).

8. The cytokine engager of claim 1, wherein the cytokine and/or the second cytokine is each an IL-2 mutant comprising one or more of the following substitutions:
E61K, E61Q, E61A, E62A, E62K, E62Q, H16A, H16E, H16Q, D20A, D20T, F42A, F42K, R38A, R38E, L72G, L72A, N88R, N88A, C125A, C125S, C125V, Q126A, or Q126T.

9. The cytokine engager of claim 1, wherein the cytokine and/or the second cytokine is an IL-2 comprising an E61K substitution.

10. (canceled)

11. The cytokine engager of claim 7, wherein the IL-2 does not comprise SEQ ID NO: 15.

12. The cytokine engager of claim 7, wherein the IL-2 comprises a sequence according to any one of SEQ ID NOs: 12 or 17-39.

13. (canceled)

14. The cytokine engager of claim 1, wherein the antibody comprises a variable heavy chain and a variable light chain comprising the amino acid sequence of SEQ ID NO: 2 and SEQ ID NO: 1, respectively.

15. The cytokine engager of claim 1, wherein the antibody comprises an Fc region from an IgG antibody, and wherein the Fc region is from an IgG1 antibody.

16. (canceled)

17. The cytokine engager of claim 1, wherein the antibody comprises an Fc region comprising the mutations L234F, L235E, and P331S, wherein the numbering is according to the EU index as in Kabat.

18. A cytokine engager, wherein each heavy chain of the antibody comprises SEQ ID NO: 4 and each light chain of the antibody comprises SEQ ID NO: 3.

19. The cytokine engager of claim 18, wherein each heavy chain of the antibody comprises SEQ ID NO: 5 and each light chain of the antibody comprises SEQ ID NO: 3.

20. The cytokine engager of claim 1, wherein the cytokine engager binds to CD8⁺ T cells and promotes proliferation of the CD8⁺ T cells.

21. (canceled)

22. The cytokine engager of claim 1, wherein the cytokine engager promotes the expression of CD69 of CD8⁺ T cells.

23. The cytokine engager of claim 22, wherein the cytokine engager induces the expression of CD69 in CD8⁺ T cells 2-fold, 5-fold, or greater than 5-fold higher than the expression of CD69 in NK cells.

24. A pharmaceutical composition comprising the cytokine engager of claim 1 and a pharmaceutically acceptable carrier.

25. An isolated nucleic acid sequence encoding the cytokine engager of claim 19.

26. A vector comprising the isolated nucleic acid sequence of claim 25.

27. A method of expanding T cells comprising contacting the pharmaceutical composition of claim 24, to a population of T cells.

28. The method of claim 27, wherein the cytokine engager is administered to a subject in need.

29. The method of claim 28, wherein the population of T cells is endogenous to the subject.

30. The method of claim 28, wherein the subject has cancer.

31. The method of claim 27, wherein the CD8⁺ T cells preferentially expand compared to CD4⁺ T cells.

32. The method of claim 27, wherein the CD8⁺ T cells preferentially expand compared to NK cells.

33. The method of claim 31, wherein the expansion is compared to contacting SEQ ID NO: 12 to a population of T cells.

34. (canceled)

35. (canceled)

36. (canceled)

37. The method of claim 30, wherein the cancer is melanoma, renal cell carcinoma, gastric, or non-small cell lung cancer.

38. A cytokine engager comprising an antibody that specifically binds cluster of differentiation 8 (CD8), wherein the antibody specifically binds to the CD8α chain of CD8, and wherein the antibody comprises a heavy chain complementarity determining region (HCDR) 1, a HCDR2, and a HCDR3, comprising the amino acid sequence of SEQ ID NO: 6, SEQ ID NO: 7, and SEQ ID NO: 8, respectively; and a light chain complementarity determining region (LCDR) 1, a LCDR2, and a LCDR3, comprising the amino acid sequence of SEQ ID NO: 9, SEQ ID NO: 10, and SEQ ID NO: 11, respectively, wherein the antibody is linked to a cytokine comprising a first heavy chain, a second heavy chain, and two light chains, wherein:
a. either the first heavy chain or the second heavy chain of the antibody is fused at the C-terminus to the cytokine; or
b. the first heavy chain is fused at the C-terminus to the cytokine and the second heavy chain of the antibody is fused at the C-terminus to a second cytokine,
wherein the antibody is fused to the cytokine or the second cytokine via a linker comprising SEQ ID NO: 13 or SEQ ID NO: 14, and wherein the cytokine or the second cytokine is interleukin-2 (IL-2).

39. A method of treating cancer comprising administrating a patient in need thereof the cytokine engager of claim 38.
